(19) United States
(12) Patent Application Publication
(10) Pub. No.: US 2004/0115176 A1
(43) Pub. Date: Jun. 17, 2004

(54) Fibrin-based tissue-engineered vasculature

(76) Inventors: Daniel D. Swartz, Attica, NY (US); Stelios T. Andreadis, Williamsville, NY (US)

Correspondence Address: Nixon Peabody LLP, Clinton Square, P.O. Box 31051, Rochester, NY, 14603-1051, US

(21) Appl. No.: 10/692,381

(22) Filed: Oct. 23, 2003

Related U.S. Application Data
(60) Provisional application No. 60/421,015, filed on Oct. 23, 2002.

(51) Int. Cl. A61K 45/00; C12N 5/08

(52) U.S. Cl. 424/93.7; 435/366

(57) ABSTRACT

A method of producing a tissue-engineered vascular vessel by providing a vessel-forming mixture of fibrinogen, thrombin, and cells, molding the vessel-forming mixture into a fibrin gel having a tubular shape, and incubating the fibrin gel in a medium suitable for growth of the cells. The resulting tissue-engineered vascular vessel and a method of producing a tissue-engineered vascular vessel for a particular patient are also disclosed.

This application claims the benefit of U.S. Provisional Patent Application Serial No. 60/421,015, filed Oct. 23, 2002, which is hereby incorporated by reference in its entirety.

### FIELD OF THE INVENTION

The present invention relates to tissue-engineered vasculature and methods of producing tissue-engineered vasculature.

### BACKGROUND OF THE INVENTION

Vascular disease involving atherosclerosis such as coronary artery disease and peripheral vascular disease is currently the largest cause of death in western developed countries (American Heart Association. 2000 Heart and Stroke Statistical Update. Dallas, Tex., USA., American Heart Association). There is currently much research being done not only looking at prevention but also the treatment of vascular disease. At present, replacement of diseased vasculature is an approach which is frequently employed, but is highly hindered by the unavailability of suitable vasculature replacement and the lack of long term success.

Many approaches have been taken to replace diseased or damaged blood vessels within the body. Synthetic conduits have been used extensively with a great degree of success in the replacement of large diameter (>6 μm) vessels. These conduits are primarily composed of expanded polytetra-flouroethylene (Teflon, ePTFE) or polyethylene terephthalate (Dacron™) (Szilagyi et al., Journal of Vascular Surgery 3(3):421-36 (1986)). However, there has been demonstrated a high failure rate of the synthetic grafts when replacing small diameter vessels due to thrombus and plaque formation. To address this issue of failure, proteins or cells, such as endothelial cells, have been added to the luminal surface to aid in limiting thrombus and plaque formation (Drury et al., Annals of Vascular Surgery 1(5):542-7 (1987); Freischlag et al., Annals of Vascular Surgery 4(5):449-54 (1990); Williams et al., Journal of Biomedical Materials Research 28(2):203-12 (1994); Pasic et al., Circulation 92(9):2605-16 (1995)). These biosynthetic grafts show a slight decrease in the failure rate, but still lack reactivity and long term patency. Allografts (grafts taken from other humans) have been used quite extensively. These grafts have demonstrated long term patency and reactivity, but immunogenic response is high. Autografts (grafts taken from one's own body) are currently the most widely used for small diameter vessel replacement; saphenous veins and radial arteries are used predominantly in coronary artery bypass procedures. The greatest limitations of autologous grafts are limited availability, especially for repeat grafting procedures, and the pain and discomfort associated with the donor site.

The development of a tissue-engineered small-diameter vascular graft has been approached in four distinct ways. All of these approaches follow the general guideline of using no permanent synthetic material. First, the acellular approach involves implanted decellularized tissues that are cellularized from the host. These tissues may be modified to enhance biocompatibility, strength, cellular adhesion, and ingrowths (Huynh et al., Nature Biotechnology 17(11):1083-6 (1999); Bader et al., Transplantation 70(1):7-14 (2000)). Unlike this method, the other three methods involve the addition of cells to the construct prior to implantation. The second approach is self-assembly, where cells are grown on plastic and induced to secrete high amounts of extracellular matrix (L'Heureux et al., FASEB Journal 12(1):47-56 (1998); L'Heureux et al., FASEB Journal 15(2):515-24 (2001); Hoerstrup et al., ASAIO Journal 48(3):234-8 (2002)). Cell sheets are formed that can later be removed from culture and then wrapped around a mandrel to form multilayer tubes. The next two methods rely on the use of biodegradable polymer scaffolds. The third approach, the cell-added synthetic matrix, involves adding cells to preformed structures made of biodegradable polymers (Shin'oka et al., Journal of Thoracic & Cardiovascular Surgery 115(3):536-45 (1998); Niklason et al., Science 284(5413):489-93 (1999); Shin'oka et al., New England Journal of Medicine 344(7):532-3 (2001); Niklason et al., J Vasc Surg 33(3):628-38 (2001); Hoerstrup et al., European Journal of Cardio-Thoracic Surgery 20(1):164-9 (2001)). This method depends on cell invasion or cell induced migration and attachment to the polymer surface.

The final approach is cell entrapment within a biopolymer, which involves the use of gels, typically type 1 collagen, which is molded into a tube after cells are added to the solution phase prior to gelation (Weinberg and Bell Science 231(4736):397-400 (1986); L'Heureux et al., Journal of Vascular Surgery 17(3):499-509 (1993)). When the cells compact these gels and an appropriate mechanical constraint is applied, it yields a circumferential alignment of fibrils and cells which resemble that of the vascular media (L'Heureux et al., Journal of Vascular Surgery 17(3):499-509 (1993); Barocas et al., J Biomech Eng 120(5):660-6 (1998); Seliktar et al., Annals of Biomedical Engineering 28(4):351-62 (2000); Girton et al., Journal of Biomechanical Engineering 124(5):568-75 (2002)). This alignment characteristic is very important in the development of the functionality of the vasculature. Mechanical function is dependent on structure, interactions of cells and extracellular matrix (alignment), equally to that of composition. Function is also important in the remodeling of the tissue-engineered vascular vessels. The structure-function relationship provides a template for the vessel as remodeling occurs.

The four methods used in the development of a tissue-engineered small-diameter vascular graft are similar in the intended outcome of vascular development. They all require the adherence of cells within the matrix to form contiguous tissue that remodels to become compatible with the environment. As the initial matrix scaffold is replaced by cell-derived secreted extracellular matrix, the vasculature demonstrates biocompatibility.

Determining the appropriate scaffold material to use is an important step in tissue engineering. The use of native decellularized tissues such as deepidermalized dermis or small intestine submucosa is limited by the efficiency of seeding and the time frame required to recellularize the tissue to a point of functionality. Biodegradable polylactides or various copolymers have been used quite extensively (Niklason et al., Science 284(5413):489-93 (1999); Hoerstrup et al., Circulation 102(19 Suppl 3):III44-9) (2000)). These synthetic polymers have good strength, large void volumes, controlled degradation and have low immunogenicity. Constructs of low reactivity and high tensile strength have been produced. The drawbacks of these polymers are poorly controlled degradation rates and the poor maturation of cells and tissue in close proximity to the polymer matrix material. In addition, these polymers take as long as 8 weeks to attain this level of function (Niklason et al., Science 284(5413):489-93 (1999)). The use of copolymers of lactic and glycolic acid is disadvantaged by their bulk degradation and effects of inhibition on vascular smooth muscle cells (“VSMC”s). They have poor seeding efficiencies and their degradation products produce lactic acid which has profound negative local pH effects. Furthermore, the degradation patterns of the copolymers disrupt tissue continuity and strength.

Collagen gels demonstrate some advantages of seeding efficiency, including uniform cell distribution and cellular alignment. However, collagen does not stimulate VSMC secretion of extracellular matrix, nor does it demonstrate development resulting in sufficient strength and function. Weinberg and Bell (Science 231(4736):397-400 (1986)), used a collagen gel because of collagen's major role in native vessels for structural strength and the major component of the tissue's extracellular matrix. The luminal surface was coated with endothelial cells and the media was comprised of smooth muscle cells. Prostacyclin and von Willibrand factor were produced by the endothelium which was sufficient for barrier function. However, the collagen gel failed in structural strength tests without an integrated polyester mesh and did not possess a controllable degradation. Smooth muscle cells secrete little extracellular matrix when entrapped in collagen gels (Thie et al., European Journal of Cell Biology 55(2):295-304 (1991); Clark et al., Journal of Cell Science 108(Pt 3):1251-61 (1995)).

The present invention is directed to overcoming these limitations.

### SUMMARY OF THE INVENTION

One aspect of the present invention is directed to a method of producing a tissue-engineered vascular vessel. This method involves providing a vessel-forming fibrin mixture comprised of fibrinogen, thrombin, and cells suitable for forming a vascular vessel. The vessel-forming fibrin mixture is molded into a fibrin gel having a tubular shape. The fibrin gel is then incubated in a medium suitable for growth of the cells under conditions effective to produce a tissue-engineered vascular vessel.

A second aspect of the present invention is directed to a tissue-engineered vascular vessel. The tissue-engineered vascular vessel is made of a gelled fibrin mixture comprising fibrinogen, thrombin, and cells. The gelled fibrin mixture has a tubular shape.

A third aspect of the present invention is directed to a method of producing a tissue-engineered vascular vessel for a particular patient. This method involves providing a vessel-forming fibrin mixture comprised of fibrinogen, thrombin, and cells suitable for forming a vascular vessel, at least one of which is autologous to the patient. The fibrin mixture is molded into a fibrin gel having a tubular shape and then incubated in a medium suitable for growth of the cells under conditions effective to produce a tissue-engineered vascular vessel for a particular patient. The tissue-engineered vascular vessel is then implanted into the patient.

Using a fibrin gel derived from a mixture of fibrinogen, thrombin, and cells suitable for forming a vascular vessel to develop tissue engineered vasculature has the possibility of greatly enhancing tissue-engineered vascular grafts. The use of a fibrin gel scaffold greatly enhances seeding density, biocompatibility, strength, and other essential characteristics of vasculature grafts. Thus, the methods of the present invention are directed to providing a tissue-engineered vascular vessel that is more compatible to implantation, limits immune rejection, is more functional, demonstrates the ability to remodel, is strong enough to withstand implantation, has a higher degree of vasoactive reactivity, and can be developed in a time frame that is useable.

### BRIEF DESCRIPTION OF THE DRAWINGS

FIGS. 1A-B are images showing fibrin gel tissue-engineered vessel constructs of the present invention molded from a 3.5 mg/ml fibrinogen/thrombin mixture and 1.66×10⁶ vascular smooth muscle cells per ml around a 4.0 mm silastic tubing. The fibrin constructs were cultured for two weeks in culture medium and either pulsed at a 5-10% radial distention at 60 beats/min., or not pulsed at all. The physical appearance between the two fibrin constructs is noted to be highly varied. The pulsed construct shown in FIG. 1A is longer in length with a smaller wall thickness than that of the non-pulsed construct shown in FIG. 1B. The grid is 2 cm squared.

FIGS. 2A-D are images of fibrin gel tissue-engineered vascular vessel constructs of the present invention molded from a 3.5 mg/ml fibrinogen/thrombin mixture and 1.66×10⁶ vascular smooth muscle cells per ml around a 4.0 mm silastic tubing. FIGS. 2A-B are images of vessel constructs stained with hematoxylin and eosin (“H&E Stain”). FIGS. 2C-D are images of vessel constructs stained with with Mason's Trichrome Stain. The vessel constructs in FIGS. 2A and 2C were cultured for two weeks in culture medium and pulsed at a 5-10% radial distention at 60 beats/min. The vessel constructs in FIGS. 2B and 2D were cultured for two weeks in culture medium, but were not pulsed. All four of the constructs in FIGS. 2A-D were formalin fixed and paraffin embedded. The pulsed vessel constructs (FIGS. 2A and 2C) demonstrate a higher degree of cellular alignment and cell spreading than that of the non-pulsed fibrin vessel constructs (FIGS. 2B and 2D).

FIGS. 3A-C are images of fibrin gel tissue-engineered vessel constructs of the present invention molded from a 3.5 mg/ml fibrinogen/thrombin mixture and 1.66×10⁶ vascular smooth muscle cells per ml around a 4.0 mm silastic tubing. The fibrin vessel construct of FIG. 3A was cultured for 5 days in culture medium and pulsed at a 5-10% radial distention at 60 beats/min. The fibrin vessel construct of FIG. 3B was cultured for 10 days in culture medium and pulsed at a 5-10% radial distention at 60 beats/min. The fibrin vessel construct of FIG. 3C was cultured for 15 days in culture medium and pulsed at a 5-10% radial distention at 60 beats/min. All three of the vessel constructs were formalin fixed and paraffin embedded and stained with Mason's Trichrome Stain to visualize the type I collagen and cell nuclei. With increasing time, there was an increase in cellular alignment and secretion of extracellular matrix (type I collagen).

FIGS. 4A-B are images of tissue-engineered vessel constructs of the present invention that are a composite of polylactic-glycolic acid (“PLGA”) fiber mesh and fibrin gel. FIG. 4A is an image of a vessel construct that is a composite of PLGA fiber mesh and fibrin gel using an H&E stain. FIG. 4B is an image of a vessel construct that is a composite of PLGA fiber mesh and fibrin gel using a Mason's Trichrome Stain. The vascular smooth muscle cells were added into the fibrin gel which was applied to the PLGA fiber mesh prior to gelation. The constructs were cultured for 4 weeks in medium containing 20 μg/ml aprotinin, at which time formalin was fixed and paraffin was embedded. The images show the distribution of cells within the constructs as well as the type I collagen secretion. The surface toward the lumen (silastic tubing) shows a loose structure with few cells or matrix deposition. The fibrin gel and cells were added from the outer surface.

FIG. 5 is a graph showing total weight of tissue-engineered vessel constructs of the present invention developed under non-pulsed conditions and varying concentrations of aprotinin at a two week time period. There was an increase in total weight of the vessel constructs with an increase in aprotinin concentration. * indicates p<0.05 as compared to 0 μg/ml aprotinin.

FIG. 6 is a graph showing total weight of tissue-engineered vessel constructs of the present invention developed under pulsed conditions and varying concentrations of aprotinin (0, 10, 20, and 200 μg/ml) at a two week time period. The pulsation for 0, 10, 20, and 200 μg/ml aprotinin was continuous at a 10% distention at 60 beats per minute starting at 48 hours. The pulsation for the 20ap group (20 μg/ml aprotinin, altered pulsation) was at an interval of 1 hour per 12 hours starting at 48 hours. There was an increase in total weight with increase in aprotinin concentration with the altered pulsation being greater than the continuous pulsation at the same 20 μg/ml of aprotinin. * indicates p<0.05 as compared to 0 μg/ml aprotinin. † indicates p<0.05 as compared to 20 μg/ml aprotinin.

FIG. 7 is a graph showing total weight of tissue-engineered vessel constructs of the present invention developed under pulsed and non-pulsed conditions, and at varying concentrations of aprotinin (0, 10, 20, and 200 μg/ml) at a two week time period. The pulsation for 0, 10, 20, and 200 μg/ml aprotinin was continuous at a 10% distention at 60 beats per minute starting at 48 hours. The pulsation for the 20ap group (20 μg/ml aprotinin, altered pulsation) was at an interval of 1 hour per 12 hours starting at 48 hours. There was a steady or increase in total weight of the vessel constructs with increasing aprotinin concentration, with the 20ap group being greater than both other groups at 20 μg/ml aprotinin. * indicates p<0.05 as compared to pulsed constructs of the same aprotinin concentration.

FIG. 8 is a graph showing the results of a hydroxyproline assay used to calculate the collagen content of tissue-engineered vessel constructs of the present invention developed under pulsed conditions and with varying concentrations of aprotinin (0, 10, 20, and 200 μg/ml) for a two week time period. The pulsation for the 20-Ap group (20 μg/ml aprotinin, altered pulsation) was at an interval of 1 hour per 12 hours starting at 48 hours. “Ctrluv” was a native umbilical vein control. “Ctrlua” was a native umbilical artery control. The x-axis represents the amount of aprotinin added to the medium (μg/ml). Collagen content was calculated as μg/vessel construct dry weight (mg). Hydroxyproline was calculated to be 12.5% of collagen content. Data are presented as mean±SE (standard error). * indicates p<0.05 as compared to 10 μg/ml aprotinin.

FIG. 9 is a graph showing the results of a hydroxyproline assay used to calculate the collagen content of tissue-engineered vessel constructs of the present invention developed under pulsed and non-pulsed conditions and at varying concentrations of aprotinin (0, 10, 20, and 200 μg/ml) for a two week time period. The pulsation for 0, 10, 20, and 200 μg/ml was continuous at a 10% distention at 60 beats per minute starting at 48 hours. The pulsation for the AP group (20 μg/ml aprotinin, altered pulsation) was at an interval of 1 hour per 12 hours starting at 48 hours. Control tissues were native umbilical veins (ctrluv) and umbilical arteries (ctrlua). * indicates p<0.05 as compared to pulsed constructs of the same aprotinin concentration.

FIG. 10 is a graph showing the results of a hydroxyproline assay used to calculate the collagen content of tissue-engineered vessel constructs of the present invention developed under pulsed and non-pulsed conditions at 20 μg/ml aprotinin from 2 to 8 weeks time. * indicates p<0.05 as compared to pulsed vessel constructs of the same aprotinin concentration and time.

FIGS. 11A-D are images showing the proliferation of cells within tissue-engineered vessel constructs of the present invention at one week and at two weeks. Proliferating cell nuclear antigen (“PCNA”) was used to identify proliferating cells within the vessel constructs. FIGS. 11A-B are images showing cell proliferation in the constructs after 1 week. In FIG. 11A, the constructs were not pulsed. In FIG. 11B, the constructs were pulsed. FIGS. 11C-D are images showing cell proliferation in the constructs after 2 weeks. In FIG. 11C, the constructs were not pulsed. In FIG. 11D, the constructs were pulsed.

FIG. 12 is a graph showing the results of an experiment wherein PCNA staining was used to identify and quantitate the percentage of proliferating cells within the tissue-engineered vessel constructs of the present invention developed under non-pulsed conditions and varying concentrations of aprotinin (0, 10, 20, and 200 μg/ml) at a two week time period. The x-axis represents the amount of aprotinin added to the medium (μg/ml). Percentage of proliferating cells is calculated by dividing the number of proliferating cells by the total number of cells in a high power field. Data are presented as mean±SE (standard error). * indicates p<0.05 as compared to 0 μg/ml aprotinin.

FIG. 13 is a graph showing the results of an experiment wherein PCNA staining was used to identify and quantitate the percentage of proliferating cells within tissue-engineered vessel constructs of the present invention developed under pulsed conditions and varying concentrations of aprotinin (0, 10, 20, and 200 μg/ml) for a two week time period. The x-axis represents the amount of aprotinin added to the medium (μg/ml). Percentage of proliferating cells was calculated by dividing the number of proliferating cells by the total number of cells in a high power field. Data are presented as mean±SE (standard error). * indicates p<0.05 as compared to 0 μg/ml aprotinin.

FIG. 14 is a graph showing the results of an experiment wherein PCNA staining was used to identify and quantitate the percentage of proliferating cells within tissue-engineered vessel constructs of the present invention developed under pulsed and non-pulsed conditions at an aprotinin concentration of 20 μg/ml from one to eight weeks. Percentage of proliferating cells was calculated by dividing the number of proliferating cells by the total number of cells in a high power field. Data are presented as mean±SE (standard error). * indicates p<0.05 as compared to static.

FIG. 15 is a graph showing cell density determined by histological staining cell nuclei and counting them per area visualized within tissue-engineered vessel constructs of the present invention developed under non-pulsed conditions and varying concentrations of aprotinin (0, 10, 20, and 200 μg/ml) at a two week time period. Cell nuclei were stained with hematoxylin. The x-axis represents the amount of aprotinin added to the medium (mg/ml). Cell Density was calculated by dividing the number of total cells by the total area measured in a high power field. Data are presented as mean±SE (standard error). * indicates p<0.05 as compared to 0 μg/ml aprotinin.

FIG. 16 is a graph showing cell density determined by histological staining of cell nuclei and counting them per area visualized within tissue-engineered vessel constructs of the present invention developed under pulsed conditions and varying concentrations of aprotinin (0, 10, 20, and 200 μg/ml) for a two week time period. Cell nuclei were stained with hematoxylin. The x-axis represents the amount of aprotinin added to the medium (μg/ml). Cell Density was calculated by dividing the number of total cells by the total area measured in a high power field. Data are presented as mean±SE (standard error). * indicates p<0.05 as compared to 0 μg/ml aprotinin.

FIG. 17 is a graph showing cell density determined by histological staining of cell nuclei and counting them per area visualized within tissue-engineered vessel constructs of the present invention developed under pulsed and non-pulsed conditions and varying concentrations of aprotinin (0, 10, 20, and 200 μg/ml) for a two week time period. Cell nuclei were stained with hematoxylin. The x-axis represents the amount of aprotinin added to the medium (μg/ml). Cell Density was calculated by dividing the number of total cells by the total area measured in a high power field. Data are presented as mean±SE (standard error). * indicates p<0.05 as compared to pulsed constructs of the same aprotinin concentration.

FIG. 18 is a graph showing cell density determined by histological staining of cell nuclei and counting them per area visualized within tissue-engineered vessel constructs of the present invention developed under pulsed and non-pulsed conditions and varying concentrations of aprotinin (0, 10, 20, and 200 μg/ml) over an eight week time period. Cell nuclei were stained with hematoxylin. The x-axis represents the amount of aprotinin added to the medium (μg/ml). Cell Density was calculated by dividing the number of total cells by the total area measured in a high power field. Data are presented as mean±SE (standard error). * indicates p<0.05 as compared to pulsed constructs of the same time. † indicates p<0.05 as compared to 1 week.

FIG. 19 is an image of a tissue chamber, which is a modified Ussing Chamber that provides a sided system for independent flow and media exposure. The tissue chambers were placed in series with a heating block, gas exchanger/media bottle, and rotary pump that provided a stable controlled environment (max. 140 days).

FIG. 20 is a graph showing the total weight of tissue-engineered vessel constructs of the present invention developed under pulsed and non-pulsed conditions at 20 μg/ml aprotinin from 2 to 8 weeks time. Construct weight remained unchanged for the non-pulsed over time while the pulsed was elevated at 3 weeks and then returned to the same level as the non-pulsed by 8 weeks. * indicates p<0.05 as compared to pulsed constructs of the same aprotinin concentration and time. † indicates p<0.05 as compared to 1 week.

FIG. 21 is a graph showing the results of a hydroxyproline assay used to calculate the collagen content of the tissue-engineered vessel constructs of the present invention developed under non-pulsed conditions and varying concentrations of aprotinin (0, 10, 20, and 200 μg/ml) for a two week time period. The x-axis represents the amount of aprotinin added to the medium (μg/ml). Collagen content was calculated as μg/vessel construct dry weight (mg). Hydroxyproline was calculated to be 12.5% of collagen content. Data are presented as mean±SE (standard error). * indicates p<0.05 as compared to 0 μg/ml aprotinin.

FIG. 22 is a graph showing the results of an experiment wherein PCNA staining was used to identify and quantitate the percentage of proliferating cells within tissue-engineered vessel constructs of the present invention developed under pulsed and non-pulsed conditions and at varying concentrations of aprotinin (0, 10, 20, and 200 μg/ml) for a two week time period. At 0, 10, 20, and 200 μg/ml aprotinin, the pulsed group was pulsed continuously. The 20ap group was pulsed at a periodic interval of 1 hour per 12 hours. The x-axis represents the amount of aprotinin added to the medium (μg/ml). Percentage of proliferating cells was calculated by dividing the number of proliferating cells by the total number of cells in a high power field. Data are presented as mean±SE (standard error). * indicates p<0.05 as compared to pulsed constructs of the same aprotinin concentration.

FIG. 23 is a graph showing maximal constriction determined by adding 118 mM KCl to tissue-engineered vessel constructs of the present invention developed under non-pulsed conditions and varying concentrations of aprotinin for a two week time period. The x-axis represents the amount of aprotinin added to the medium (μg/ml). With increasing amounts of aprotinin, there was a decreased contractile response to 118 mM KCl. Data are presented as mean±SE (standard error). * indicates p<0.05 as compared to 0 μg/ml aprotinin.

FIG. 24 is a graph showing maximal constriction, which was determined by adding 118 mM KCl to tissue-engineered vessel constructs of the present invention developed under pulsed conditions (continuous and 1/12 (20ap)) and varying concentrations of aprotinin (0, 10, 20, and 200 μg/ml) for a two week time period. The x-axis represents the amount of aprotinin added to the medium (μg/ml). With increasing amounts of aprotinin, there was a decrease in contractile response to 118 mM KCl. Data are presented as mean±SE (standard error). * indicates p<0.05 as compared to 10 μg/ml aprotinin. † indicates p<0.05 as compared to 20 μg/ml aprotinin.

FIG. 25 is a graph showing maximal constriction, which was determined by adding 118 mM KCl to tissue-engineered vessel constructs of the present invention developed under non-pulsed and pulsed conditions (continuous and 1/12 (20ap)) and at varying concentrations of aprotinin (0, 10, 20, and 200 μg/ml) for a two week time period. The x-axis represents the amount of aprotinin added to the medium (μg/ml). With increasing amounts of aprotinin, there was a decrease in contractile response to 118 mM KCl. Data are presented as mean±SE (standard error). * indicates p<0.05 as compared to pulsed constructs of the same aprotinin concentration. † indicates p<0.05 as compared to static 20 μg/ml aprotinin.

FIG. 26 is a graph showing maximal constriction, which was determined by adding 118 mM KCl to tissue-engineered vessel constructs of the present invention developed under non-pulsed and pulsed conditions (continuous and 1/12 (20ap)) and 20 μg/ml of aprotinin over an eight week time period. The x-axis represents the number of weeks. With increasing time there was a slight increase in contractile response to 118 mM KCl for the non-pulsed constructs and a slight decrease for the pulsed constructs with the two groups differing at all time points but 2 weeks. Data are presented as mean±SE (standard error). * indicates p<0.05 as compared to pulsed constructs at the same time point.

FIG. 27 is a graph showing constriction, which was determined by adding 10⁻⁶ M norepinephrine to tissue-engineered vessel constructs of the present invention developed under non-pulsed conditions and varying concentrations of aprotinin (0, 10, 20, and 200 μg/ml) at a two week time period. The x-axis represents the amount of aprotinin added to the medium (μg/ml). With increasing amounts of aprotinin, there was a decrease in contractile response to norepinephrine. Data are presented as mean±SE (standard error). * indicates p<0.05 as compared to 0 μg/ml aprotinin.

FIG. 28 is a graph showing constriction of tissue-engineered vessel constructs of the present invention determined by adding 10⁻⁶ M norepinephrine to constructs developed under pulsed (continuous or 1/12 (20ap)) conditions and varying concentrations of aprotinin (0, 10, 20, and 200 μg/ml) at a two week time period. The x-axis represents the amount of aprotinin added to the medium (μg/ml). With increasing amounts of aprotinin, there was a decrease in contractile response to norepinephrine. Data are presented as mean±SE (standard error). * indicates p<0.05 as compared to 10 μg/ml aprotinin.

FIG. 29 is a graph showing constriction of vessels determined by adding U46619 (10⁻⁷ M), a thromboxane mimetic, into the isolated tissue bath to tissue-engineered vessel constructs of the present invention developed under non-pulsed conditions and varying concentrations of aprotinin (0, 10, 20, and 200 ug/ml) at a two week time period. The x-axis represents the amount of aprotinin added to the medium (μg/ml). With increasing amounts of aprotinin there was a decrease in contractile response to U46619. Data are presented as mean±SE (standard error). * indicates p<0.05 as compared to 0 μg/ml aprotinin.

FIG. 30 is a graph showing constriction determined by adding U46619 (10⁻⁷ M), a thromboxane mimetic, into the isolated tissue bath to tissue-engineered vessel constructs of the present invention developed under pulsed (continuous or 1/12 (20ap)) conditions and varying concentrations of aprotinin (0, 10, 20, and 200 μg/ml) at a two week time period. The x-axis represents the amount of aprotinin added to the medium (μg/ml). With increasing amounts of aprotinin there was a decrease in contractile response to U46619. Data are presented as mean±SE (standard error). * indicates p<0.05 as compared to 0 μg/ml aprotinin.

FIG. 31 is a graph showing constriction of vessels determined by adding norepinephrine (10⁻⁶ M) to tissue-engineered vessel constructs of the present invention developed under non-pulsed and pulsed conditions (continuous and 1/12 (20ap)) and varying concentrations of aprotinin (0, 10, 20, and 200 μg/ml) for a two week time period. The x-axis represents the amount of aprotinin added to the medium (μg/ml). With increasing amounts of aprotinin, there was a decrease in contractile response to 118 mM KCl. Data are presented as mean±SE (standard error). * indicates p<0.05 as compared to pulsed constructs of the same aprotinin concentration. † indicates p<0.05 as compared to static 20 μg/ml aprotinin.

FIG. 32 is a graph showing constriction determined by adding U46619 (10⁻⁶ M), a thromboxane mimetic, to tissue-engineered vessel constructs of the present invention developed under non-pulsed and pulsed conditions (continuous and 1/12 (20ap)) and varying concentrations of aprotinin at a two week time period. The x-axis represents the amount of aprotinin added to the medium (μg/ml). With increasing amounts of aprotinin, there is a decreased contractile response to U46619 in both the pulsed and non-pulsed constructs. Data are presented as mean±SE (standard error). * indicates p<0.05 as compared to pulsed constructs of the same aprotinin concentration. † indicates p<0.05 as compared to static 20 μg/ml aprotinin.

FIG. 33 is a graph showing constriction determined by adding norepinephrine (10⁻⁶ mM) to tissue-engineered vessel constructs of the present invention developed under non-pulsed and pulsed conditions (continuous and 1/12 (20ap)) and 20 μg/ml of aprotinin over an eight week time period. The x-axis represents the number of weeks. With increasing time, there is a decrease in contractile response to norepinephrine with the two groups differing at all time points but 2 weeks. Data are presented as mean±SE (standard error). * indicates p<0.05 as compared to pulsed constructs at the same time point.

FIG. 34 is a graph showing vessel constriction determined by adding U46619 (10⁻⁷M), a thromboxane mimetic, to tissue-engineered vessel constructs of the present invention developed under non-pulsed and pulsed conditions (continuous and 1/12 (20ap)) and 20 μg/ml of aprotinin over an eight week time period. The x-axis represents the number of weeks. With increasing time, there was a decrease in contractile response to U46619, with the two groups differing at all time points but 2 weeks. Data are presented as mean±SE (standard error). * indicates p<0.05 as compared to pulsed constructs at the same time point.

FIG. 35 is a graph showing vessel relaxation to SNAP, a sodium nitroprusside derivative-nitric oxide donor, of a norepinephrine constriction. Relaxation was reported as a percent of the NE constriction at 2 week time point. Tissue-engineered vessel constructs of the present invention were non-pulsed at 0, 10, 20, and 200 μg/ml aprotinin. Reported concentrations of SNAP are 10⁻⁷M and 10⁻⁶M. * indicates p<0.05 as compared to 0 μg/ml aprotinin. † indicates p<0.05 as compared to previous concentration.

FIG. 36 is a graph showing stretch length at 1 gram of tension of tissue-engineered vessel constructs of the present invention developed under non-pulsed conditions and varying concentrations of aprotinin (0, 10, 20, and 200 μg/ml) at a two week time period. There was a small decrease in stretch length with increasing aprotinin concentration. * indicates p<0.05 as compared to 0 μg/ml aprotinin.

FIG. 37 is a graph showing vessel stretch length at 1 gram of tension of tissue-engineered vessel constructs of the present invention developed under pulsed conditions and varying concentrations of aprotinin (0, 10, 20, and 200 μg/ml) at a two week time period. The pulsation for 0, 10, 20, and 200 μg/ml aprotinin was continuous at a 10% distention at 60 beats per minute starting at 48 hours. The pulsation for the 20ap group was at an interval of 1 hour per 12 hours starting at 48 hours. There was a small decrease in stretch length with increasing aprotinin concentration. * indicates p<0.05 as compared to 10 μg/ml aprotinin.

FIG. 38 is a graph showing stretch length at 1 gram of tension of tissue-engineered vessel constructs of the present invention developed under pulsed and non-pulsed conditions and varying concentrations of aprotinin (0, 10, 20, and 200 μg/ml) at a two week time period. The pulsation for 0, 10, 20, and 200 μg/ml was continuous at a 10% distention at 60 beats per minute starting at 48 hours. The pulsation for the 20ap group was at an interval of 1 hour per 12 hours starting at 48 hours. There was a small decrease in stretch length with increasing aprotinin concentration, and stretch length was always greater in the pulsed constructs compared to the non-pulsed. * indicates p<0.05 as compared to pulsed constructs of the same aprotinin concentration.

FIG. 39 is a graph showing measurements of stretch lengths at 1 gram of tension of tissue-engineered vessel constructs of the present invention developed under pulsed and non-pulsed conditions at aprotinin concentration of 20 μg/ml from 2 to 8 weeks time. Construct length is greater under pulsed conditions at all time points after 1 week. * indicates p<0.05 as compared to pulsed constructs of the same aprotinin concentration and time.

FIG. 40 is a graph showing maximal break length in tissue-engineered vessel constructs of the present invention determined by placing the constructs, which were developed under non-pulsed conditions and varying concentrations of aprotinin (0, 10, 20, and 200 μg/ml) at a two week time period, into the isolated tissue bath and applying tension until the construct breaks. The break-length was then measured. As the aprotinin concentration increased from 0 to 20 μg/ml, the breaking stretch length also increased. However, from 10 to 200 μg/ml aprotinin, the breaking stretch length remained about the same. The x-axis represents the amount of aprotinin added to the medium (μg/ml). Data are presented as mean±SE (standard error). * indicates p<0.05 as compared to 0 μg/ml aprotinin.

FIG. 41 is a graph showing maximal break length in vessels determined by placing the tissue-engineered vessel constructs of the present invention, which were developed under pulsed conditions, and varying concentrations of aprotinin (0, 10, 20, and 200 μg/ml) at a two week time period, into the isolated tissue bath and applying tension until the construct breaks. The break-length was then measured. As the aprotinin concentration increased from 0 to 200 μg/ml, the breaking stretch length also increased. The x-axis represents the amount of aprotinin added to the medium (μg/ml). Data are presented as mean±SE (standard error). * indicates p<0.05 as compared to 0 μg/ml of aprotinin.

FIG. 42 is a graph showing maximal break length in vessels determined by placing the tissue-engineered vessel constructs of the present invention, which were developed under pulsed and non-pulsed conditions and varying concentrations of aprotinin (0, 10, 20, and 200 μg/ml) at a two week time period, into the isolated tissue bath and applying tension until the construct breaks, then measuring that length. As the aprotinin concentration increased from 0 to 200 μg/ml, the breaking stretch length also increased. The x-axis represents the amount of aprotinin added to the medium (μg/ml). Data are presented as mean±SE (standard error). * indicates p<0.05 as compared to 0 μg/ml aprotinin.

FIG. 43 is a graph showing maximal break length of vessels determined by placing the tissue-engineered vessel constructs of the present invention, which were developed under pulsed and non-pulsed conditions at 20 μg/ml of aprotinin at a two week time period, into the isolated tissue bath and applying tension until the construct breaks, then measuring that length. As the aprotinin concentration increased from 0 to 200 μg/ml the breaking stretch length decreased. The x-axis represents the amount of aprotinin added to the medium (μg/ml). Data are presented as mean±SE (standard error). * indicates p<0.05 as compared to pulse constructs at the same aprotinin concentration and time.

FIG. 44 is a graph showing length-tension curve generated from tissue-engineered vessel constructs of the present invention at 1 week time point comparing non-pulsed to pulsed at 20 μg/ml aprotinin. Curves are generated by incrementally increasing the tension applied to the constructs and obtaining correlating tissue stretch lengths. The regression line equation for the non-pulsed constructs was: y=−1952175+289639X; R\hat{ }2=0.966, n=2. The regression line equation for the pulsed constructs was: y=−1955414+302484X; R=0.934, n=2. There is no significant difference between the groups.

FIG. 45 is a graph showing a length-tension curve generated from tissue-engineered vessel constructs of the present invention at a 2 week time point comparing non-pulsed to pulse at 20 μg/ml aprotinin. Curves are generated by incrementally increasing the tension applied to the constructs and obtaining correlating tissue stretch lengths. The regression line equation for the non-pulsed constructs was: y==1993697+336316X; RA2=0.921, n=7. The regression line equation for the pulsed constructs was: y=−320469+54271X; R=0.707, n=6. P value<0.05.

FIG. 46 is a graph showing maximal tension determined by placing tissue-engineered vessel constructs of the present invention, which were developed under non-pulsed conditions and varying concentrations of aprotinin (0, 10, 20, and 200 μg/ml) at a two week time period, into the isolated tissue bath and applying tension until the construct breaks. With increasing amounts of aprotinin, there was an increased ability of the construct to withstand greater amounts of tension before breaking. However, at 200 μg/ml aprotinin, the maximal tension is lower than at 20 μg/ml aprotinin. The x-axis represents the amount of aprotinin added to the medium (μg/ml). Data are presented as mean±SE (standard error). * indicates p<0.05 as compared to 0 μg/ml aprotinin.

FIG. 47 is a graph showing maximal tension determined by placing tissue-engineered vessel constructs of the present invention, which were developed under pulsed (continuous and 1/12 (20ap)) conditions and varying concentrations of aprotinin (0, 10, 20, and 200 μg/ml) at a two week time period, into the isolated tissue bath and applying tension until the construct breaks. With increasing amounts of aprotinin, there was an increased ability of the construct to withstand greater amounts of tension before breaking. However, at 20ap (20 μg/ml aprotinin, altered pulsation), the maximal tension was higher than at 20 μg/ml. The x-axis represents the amount of aprotinin added to the medium (μg/ml). Data are presented as mean±SE (standard error). * indicates p<0.05 as compared to 0 μg/ml aprotinin. † indicates p<0.05 as compared to 20 μg/ml aprotinin.

FIG. 48 is a graph showing maximal tension determined by placing tissue-engineered vessel constructs of the present invention, which were developed under non-pulsed and pulsed (continuous and 1/12 (20ap)) conditions and varying concentrations of aprotinin (0, 10, 20, and 200 μg/ml) at a two week time period, into the isolated tissue bath and applying tension until the construct breaks. With increasing amounts of aprotinin, there was an increased ability of the constructs to withstand greater amounts of tension before breaking. However, this pattern of increased maximal tensile strength with increasing aprotinin concentration varies between non-pulsed and pulsed. The x-axis represents the amount of aprotinin added to the medium (μg/ml). Data are presented as mean±SE (standard error). * indicates p<0.05 as compared to pulsed of same aprotinin. † indicates p<0.05 as compared to 20 μg/ml aprotinin.

FIG. 49 is a graph showing maximal tension determined by placing tissue-engineered vessel constructs of the present invention, which were developed under non-pulsed and pulsed (continuous and 1/12 (20ap)) conditions and varying concentrations of aprotinin (0, 10, 20, and 200 μg/ml) at a two week time period, into the isolated tissue bath and applying tension until the construct breaks. The x-axis represents the number of weeks. With increasing time, there was a decrease in maximal tension for both groups, and, at 3 weeks, the maximal tension remained steady. Data are presented as mean±SE (standard error). * indicates p<0.05 as compared to pulsed constructs at the same time point.

FIG. 50 is an image of an angiogram of a lamb 5 weeks post grafting of a tissue-engineered vascular vessel of the present invention into the external jugular. The distal end of the graft was marked with a radiopaque tie. Contrast was injected from the distal end of the graft and diffused retrograde through the graft before clearing by antegrade flow. The graft appears to be partially occluded with thrombus or plaque formations. The graft was incubated for 2 weeks prior to implantation with endothelial cells seeded to the outer surface 3 days prior to implantation. The graft was inverted at time of grafting to position endothelium in the lumen.

### DETAILED DESCRIPTION OF THE INVENTION

One aspect of the present invention is directed to a method of producing a tissue-engineered vascular vessel. This method involves providing a vessel-forming fibrin mixture comprised of fibrinogen, thrombin, and cells suitable for forming a vascular vessel. The vessel-forming fibrin mixture is molded into a fibrin gel having a tubular shape. The fibrin gel is then incubated in a medium suitable for growth of the cells under conditions effective to produce a tissue-engineered vascular vessel.

A second aspect of the present invention is directed to a tissue-engineered vascular vessel. The tissue-engineered vascular vessel is made of a gelled fibrin mixture comprising fibrinogen, thrombin, and cells. The gelled fibrin mixture has a tubular shape.

A third aspect of the present invention is directed to a method of producing a tissue-engineered vascular vessel for a particular patient. This method involves providing a vessel-forming fibrin mixture comprised of fibrinogen, thrombin, and cells suitable for forming a vascular vessel, at least one of which is autologous to the patient. The fibrin mixture is molded into a fibrin gel having a tubular shape and then incubated in a medium suitable for growth of the cells under conditions effective to produce a tissue-engineered vascular vessel for a particular patient. The tissue-engineered vascular vessel is then implanted into the particular patient.

It has been discovered that fibrin gels are effective matrix scaffolds for the development of tissue-engineered vascular vessels. Fibrin gels are biodegradable and biocompatible when made from allogenic or autologous sources. Fibrin gels also support the attachment of cells to biological surfaces, enhance the migration capacity of transplanted cells, and allow diffusion of growth and nutrient factors. Cells can be seeded directly into the gel to optimize seeding efficiencies. Fibrin gels possess other favorable qualities that make them effective in tissue-engineered vasculature constructs (Ye et al., European Journal of Cardio-Thoracic Surgery 17(5):587-91 (2000); Jockenhoevel et al., European Journal of Cardio-Thoracic Surgery 19(4):424-30 (2001); Grassl et al., J Biomed Mater Res 60(4):607-12 (2002), which are hereby incorporated by reference in their entirety). For example, it has been discovered that fibrin gels can be formed from autologous fibrinogen. Another favorable quality of fibrin gels is the natural presence of a molecule which stimulates Vascular Smooth Muscle Cells (“VSMC”) to secrete extracellular matrix. Extracellular matrix is a complex aggregate of glycoproteins whose structural integrity and functional composition are important in maintaining normal tissue architecture in development and in tissue function (Meredith et al., Molecular Biology of the Cell 4(9):953-61 (1993); Lee et al., Nephrology Dialysis Transplantation 10(5):619-23 (1995), which are hereby incorporated by reference in their entirety).

Moreover, fibrin, as a scaffold, has the ability to promote cell attachment and proliferation (Bunce et al., Journal of Clinical Investigation 89(3):842-50 (1992), which is hereby incorporated by reference in its entirety). Schrenk et al., Thoracic & Cardiovascular Surgeon 35(1):6-10 (1987) (which is hereby incorporated by reference in its entirety), demonstrated the pre-treatment of d-PTFE with fibrin glue improved the attachment of endothelial cells compared to that of pre-treatment with whole blood. Fibrin has been found to be less adhesive toward platelets than other adhesive proteins, even that of fibrinogen (Dvorak et al., Laboratory Investigation 57(6):673-86 (1987); Kent et al., ASAIO Transactions 34(3):578-80 (1988), which are hereby incorporated by reference in their entirety). Small diameter vascular grafts have demonstrated a high thrombogenic response. This is believed to be primarily due to the poor adhesion and spreading of vascular endothelial cells to the luminal surface.

The fibrin gel used in the methods and vessels of the present invention is derived from a fibrin mixture comprised of fibrinogen, thrombin, and cells suitable for forming a tissue-engineered vascular vessel. Fibrinogen, thrombin, and cells suitable for forming a vascular vessel of the fibrin mixture are preferably derived from an autologous source. Preferably, the fibrinogen, and thrombin of the fibrin mixture are derived from a patient's blood.

Fibrinogen is a high molecular weight macromolecule (340 kdalton), rodlike in shape, about 50 nm in length and 3 to 6 nm thick. The central domain contains two pairs of bonding sites, A and B, which are hidden by two pairs of short peptides (fibrinopeptides A and B; FPA and FPB). The polymerization sites a and b are at the ends of the outer domains, where other sites susceptible of enzymatic crosslinking are located. Fibrinogen undergoes polymerization in the presence of thrombin to produce monomeric fibrin. This process involves the production of an intermediate alpha-prothrombin which is lacking one of two fibrinopeptide A molecules, which is then followed rapidly (four times faster), by the formation of alpha-thrombin monomer, lacking both fibrinopeptide A molecules (Ferri et al., Biochemical Pharmacology 62(12):1637-45 (2001), which is hereby incorporated by reference in its entirety). Sites A and B bind to their complimentary sites on other molecules a and b respectively. The aA interaction is responsible for linear aggregation, while the bB interaction is responsible for lateral growth of the fiber. Thrombin cleavage occurs in a particular manner, first cleaving the FPAs to form linear two-stranded, half staggered chains called profibrils. Subsequently, the FPBs are cleaved allowing the fibrils to aggregate side-by-side increasing in diameter. Fibrinogen is naturally cross linked by components found in plasma, such as protransglutaminase (factor XIII) (Siebenlist et al., Thrombosis & Haemostasis 86(5):1221-8 (2001), which is hereby incorporated by reference in its entirety). This allows for the strengthening of the fibrin gel when in the presence of plasma.

The strength of the fibrin gel adhesive component may depend on the final concentration of fibrinogen. Higher fibrinogen concentrations can be achieved by increasing the mixing ratio of the typical 1:1 (thrombin:fibrinogen) mixture of the present invention to a 1:5 mixture achieving a final concentration of 57.0 mg/ml fibrinogen.

Suitable cells of the vessel-forming fibrin mixture are vascular smooth muscle cells. Other suitable cells of the vessel-forming fibrin mixture are fibroblasts. Cells suitable for the fibrin mixture of the present invention could therefore include vascular smooth muscle cells, fibroblasts, and/or mixtures thereof. Alternatively, differentiated stem cells may be used as cells suitable to the vessel-forming fibrin mixture of the present invention. The cells in the vessel-forming fibrin mixture are preferably at a concentration within the vessel-forming fibrin mixture of about 1 to 4×10⁶ cells/ml.

Vascular smooth muscle cells are particularly suitable for the vessel-forming fibrin mixture of the present invention. The integrin alpha v beta 3 of vascular smooth muscle cells has been shown to bind the RGD-containing region of the alpha chain of fibrinogen/fibrin. As fibrinogen is cleaved by thrombin, the cleavage products of fibrinogen fragments D and E effect the migration of smooth muscle cells. Integrins (alphav beta3 and alpha5 beta!) of smooth muscle cells appear to be involved with this migration (Kodama et al., Life Sciences 71(10):1139-48 (2002), which is hereby incorporated by reference in its entirety). Smooth muscle cells have a greater rate of migration in cross linked (factor XIII) fibrin gels (Naito Nippon Ronen Igakkai Zasshi—Japanese Journal of Geriatrics 37(6):458-63 (2000), which is hereby incorporated by reference in its entirety). The greater that cells such as smooth muscle, endothelial and fibroblasts adhere to a surface, the lower the production of extracellular matrix. However, TGF-beta is thought to increase the production of extracellular matrix even on high adhesive surfaces. It has been demonstrated that fibrin stimulates the production of collagen by smooth muscle cells (Clark et al., Journal of Cell Science 108(Pt 3):1251-61 (1995); Tuan et al., Experimental Cell Research 223(1):127-34 (1996), which are hereby incorporated by reference in their entirety). Supplementation of the medium with citric acid promotes vascular smooth muscle cell secretion of collagen into the extracellular matrix (Niklason et al., Science 284(5413):489-93 (1999), which is hereby incorporated by reference in its entirety).

The vessel-forming fibrin mixture of the present invention is molded into a fibrin gel having a tubular shape. The compaction of fibrin gels is a process poorly understood. If compaction were to occur in an unconstrained system such as, in a well after being released from the surface, the cells and fibrin fibers show very little organization or alignment. However, when cells compact a fibrin gel in the presence of an appropriate mechanical constraint, a circumferential alignment of fibrils and cells results, which resembles that of the vascular media (Weinberg and Bell, Science 231(4736):397-400 (1986); L'Heureux et al., Journal of Vascular Surgery 17(3):499-509 (1993), which are hereby incorporated by reference in their entirety). This alignment characteristic is very important in the development of functionality. Mechanical function is dependent on structure, interactions of cells and extracellular matrix (alignment), equally to that of composition. Function is also important in the remodeling of the tissue-engineered vasculature vessels. Their structure-function relationship provides a template for the vessel as remodeling occurs.

Molding of the fibrin mixture is preferably carried out in a silastic tube with an inner mandrel. Fibrin gel has the ability to become aligned near a surface as the gel is formed or within the gel as it compacts due to traction exerted by entrapped cells (Tranquillo, Biochem Soc Symp 65:27-42 (1999), which is hereby incorporated by reference in its entirety). The use of a central mandrel during gelation increases circumferential alignment of the smooth muscle cells as well as the matrix. The use of a mandrel also provides a large stress on the smooth muscle cells which induces secretion and accumulation of extracellular matrix that enhances the stiffening component of the construct (Barocas et al., J Biomech Eng 120(5):660-6 (1998), which is hereby incorporated by reference in its entirety).

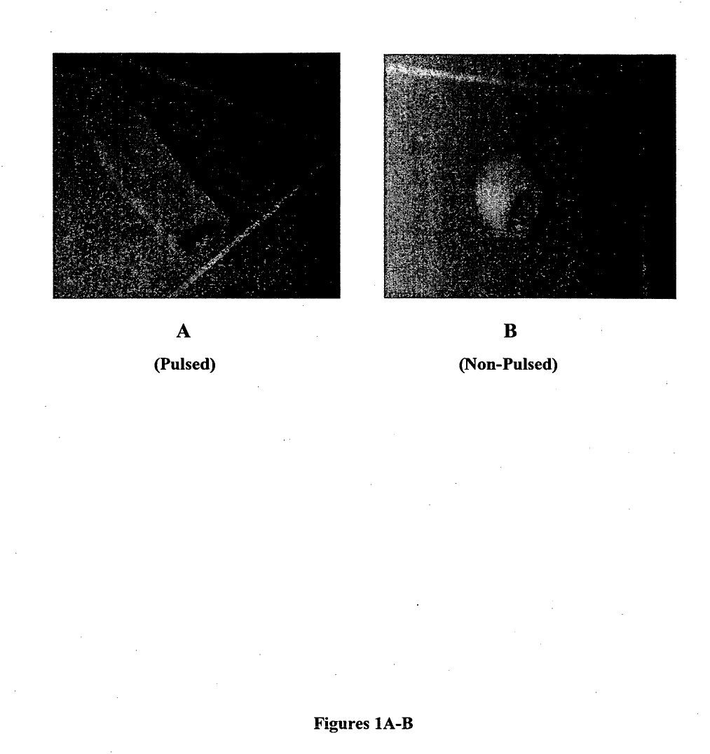

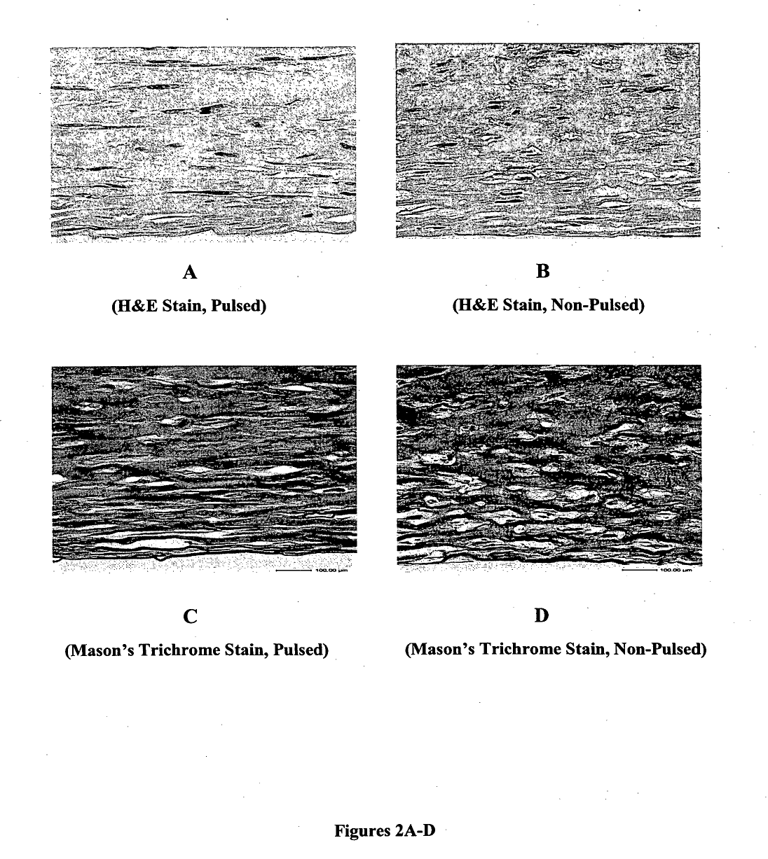

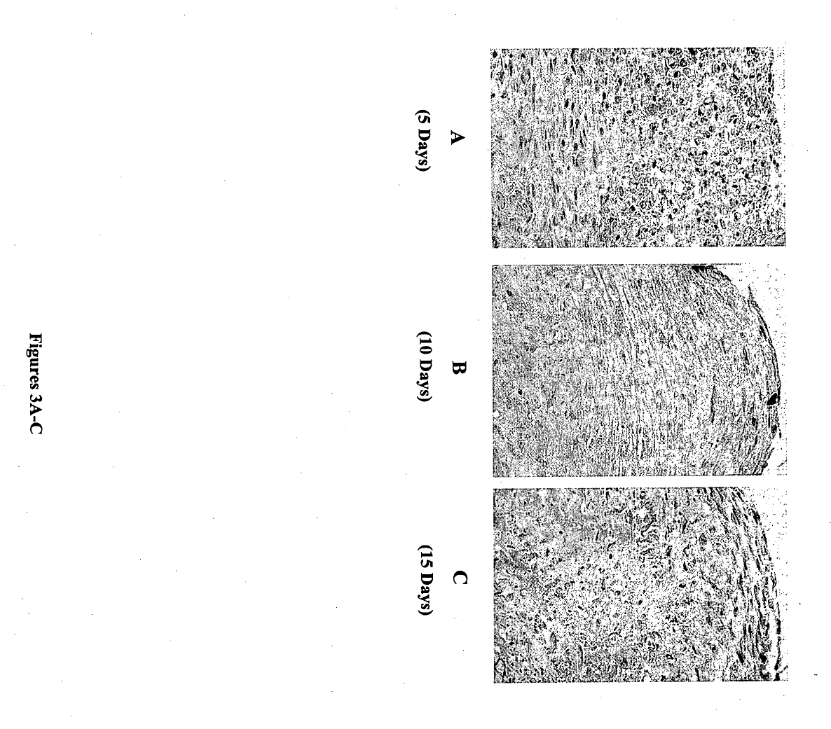

During development of the tissue-engineered vasculature of the present invention, it may be desirable to pulse the vessel constructs to modulate growth, development, and structure and/or function of the vessels. When the fibrin vessel constructs are pulsed, there is an inhibition of longitudinal compaction of the construct (FIGS. 1A-B). In the case of adding a continuous rhythmic pulsation, an increase in cellular alignment perpendicular to the applied force may be achieved (FIGS. 2A-D and FIGS. 3A-C). The increased radial alignment created from pulsation may be the limiting factor of the longitudinal compaction.

Pulsing may be achieved by applying force directly to the inner lumen of the tissue-engineered vessel constructs. For example, a roller pump may be used to pass liquid through the inner lumen of the vessels in a pulsating manner. Alternatively, the inner mandrel used in molding the vessel constructs may be connected to a pneumatic pulsation device. In some instances pulsation may have a desirable effect on the structure and/or function of the vessel. In other instances, pulsation may have a detrimental effect on the desired characteristics (structure and/or function) of the vessel.

After incubation of the fibrin gel, it is preferable to grow the cells of the fibrin mixture in a medium suitable for growth. The optimization of the fibrin gel vascular construct includes a multitude of growth factors that can be used to further development and function. In particular, high serum medias as well as keratinocyte growth factor (KGF) demonstrate an enhanced development of the fibrin gel vascular vessel construct. Also, literature cites the use of many other growth factors that have stimulated cell growth, function and behavior when used with fibrin and other gels.

A suitable medium of the present invention is comprised of M199, 1% penicillin/streptomycin, 2 mM L-glutamine, 0.25% fungizone, and 15 mM HEPES. A growth additive may also be added to the medium suitable for growth. A suitable growth additive is comprised of 50 μg/ml ascorbic acid, 10-20% FBS, 10-20 μg/ml aprotinin or 0.5-2.0 mg/ml EACA, 2 μg/ml insulin, 5 ng/ml TGFβ1, and 0.01 U/ml plasmin. In addition, a growth hormone may be included in the growth additive. Suitable growth hormones include, VEGF, b-FGF, PDGF, and KGF. Preferably, the growth medium is changed every 2-3 days.

Endothelial cells may be seeded to the interior of the tissue-engineered vascular vessel by removing the inner mandrel and seeding the cells to the interior lumen of the vessel. Cells may also be added to the outer surface of the vessels during molding. Suitable cells to be seeded to the outer surface of the vessel are fibroblasts. Alternatively, specific organ cells may be seeded to the outer surface of the tissue-engineered vascular vessel of the present invention.

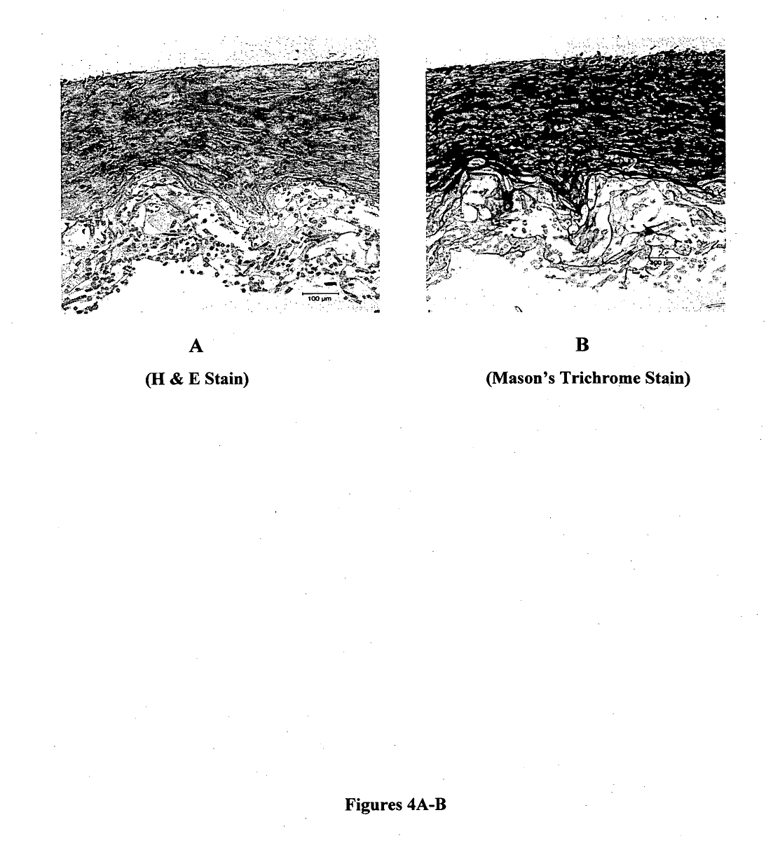

The tissue-engineered vascular vessel of the present invention may also be comprised of a fibrin gel scaffold combined with a porous scaffold to enhance vascular grafting. When the same fibrin gel, containing a uniform distribution of cells, is used in conjunction with other highly porous scaffold materials, there may be many synergistic benefits of this composite fibrin gel scaffold (FIG. 4). There are all the benefits of the fibrin gel plus the addition of early interim strength and early incorporation of other factors that may typically not be produced until later in development (elastin). Thus, the fibrin gel of the present invention can be used with any porous scaffold, such as decellularized elastin or polylactic-glycolic acid (“PLGA”) to further enhance the benefits and applicability of the fibrin gel vascular grafts. A preferable porous scaffold to be combined with fibrin gel to enhance vascular grafting is decellularized elastin. Another preferable porous scaffold to be combined with fibrin gel to enhance vascular grafting is PLGA.

Vascular smooth muscle cells are known to rapidly degrade fibrin via secretion of proteases. Thus, it is desirable to prevent this degradation during the development of the tissue-engineered vessel of the present invention. Degradation of fibrin in the vessel of the present invention can be controlled through the use of protease inhibitors. A suitable protease inhibitor of the present invention is aprotinin. In a preferred embodiment of the present invention, 0 to 200 μg/ml of aprotinin is added to the fibrin mixture to modulate fibrin degradation. Preferably, about 20 μg/ml of aprotinin is added to the fibrin mixture to modulate fibrin degradation.

Aprotinin, has the ability to slow or stop fibrinolysis. Particularly, aprotinin acts as an inhibitor of trypsin, plasmin, and kallikrein by forming reversible enzyme-inhibitor complexes (Ye et al., European Journal of Cardio-Thoracic Surgery 17(5):587-91 (2000), which is hereby incorporated by reference in its entirety). ε-aminocaproic acid (EACA), another suitable protease inhibitor of the present invention, binds plasmin to inhibit fibrinolysis (Grassl et al., J Biomed Mater Res 60(4):607-12 (2002), which is hereby incorporated by reference in its entirety). Supplementation with a protease inhibitor (epsilon-aminocaproic acid or aprotinin) to control the rate of degradation, may have a modulating effect on collagen synthesis, which is dependent on the rate of degradation (Grassl et al., J Biomed Mater Res 60(4):607-12 (2002), which is hereby incorporated by reference in its entirety). As collagen is produced, more than half appears in the medium as an aggregate with the balance retained in the matrix (Grassl et al., J Biomed Mater Res 60(4):607-12 (2002), which is hereby incorporated by reference in its entirety).

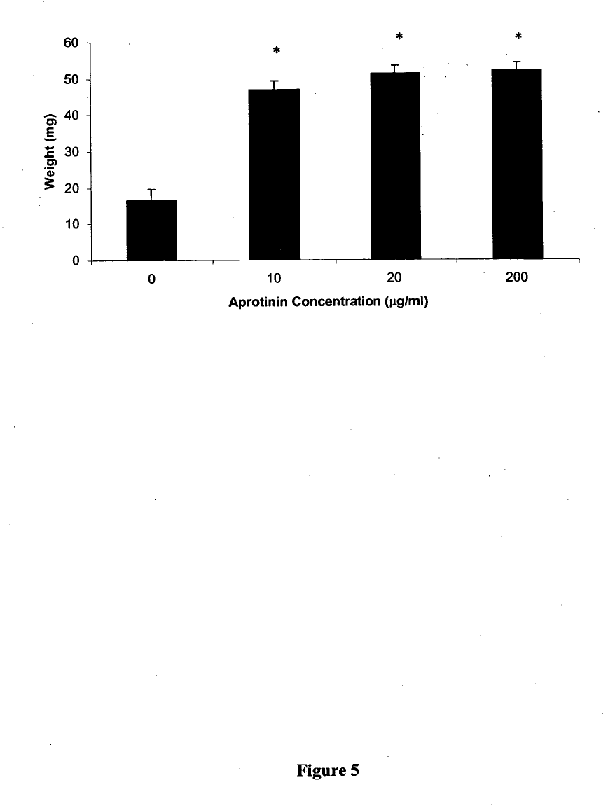

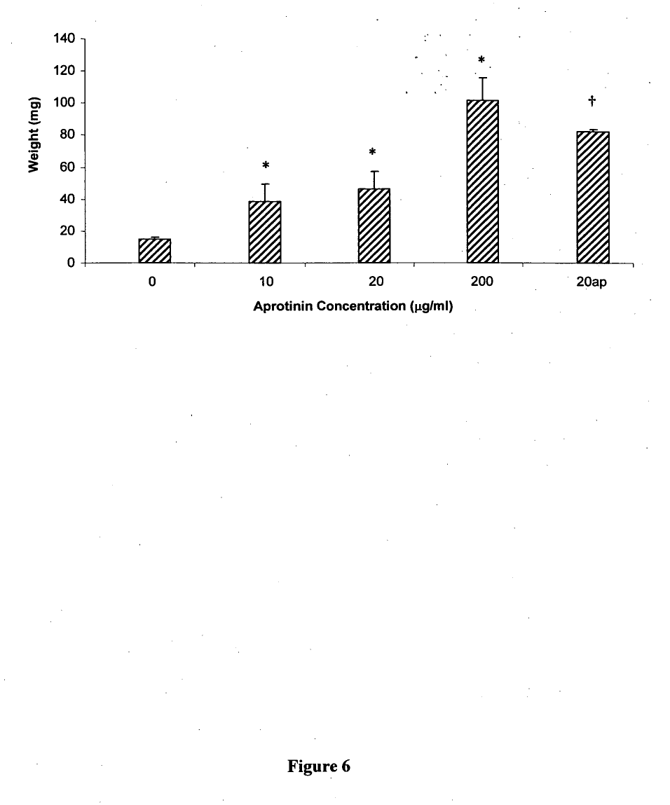

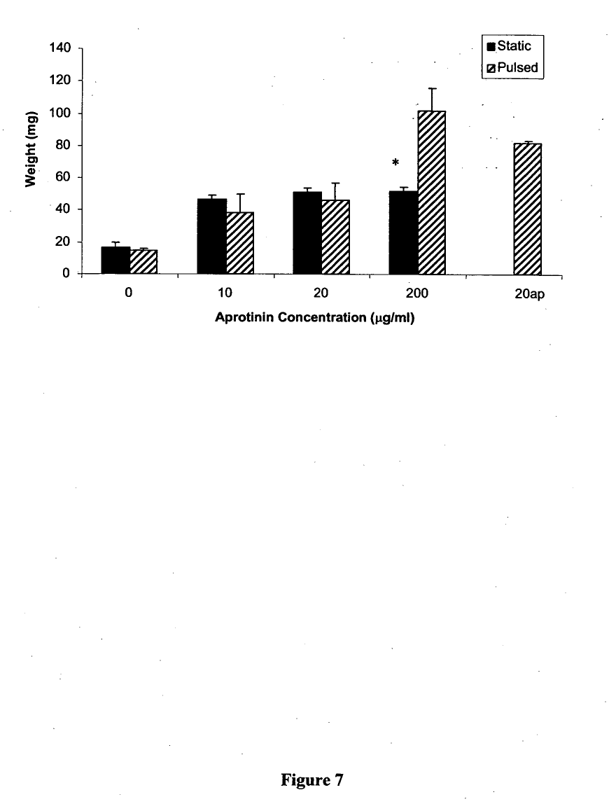

Total weight of the fibrin vessel constructs of the present invention can be affected by the amount of aprotinin added to the medium. This is evident from the increase in weight of the total vessel construct as greater amounts of aprotinin are added. However, vessel weight is not controlled totally by the addition of aprotinin because it has been observed that non-pulsed vessel weight plateaus, while pulsed vessel weight continues to rise with increasing aprotinin (FIG. 5 and FIG. 6). Thus, there appears to be a balance between secreted proteases, extracellular matrix secretion, and the added aprotinin in combination with the pulsation. The significance of the pulsation scheme is also evident from the increased vessel construct weight of the altered pulsation group from that of both groups (FIG. 7). Thus, further optimization of overall development of the tissue-engineered vascular vessels of the present invention can be obtained by adjusting the amount and degree of pulsation during development and the concentration of aprotinin.

The tissue-engineered vascular vessel of the present invention is suitable as an in vivo vascular graft. In vivo vascular grafts of the tissue-engineered vascular vessels of the present invention may be made in animals. In a preferred embodiment, the vessel is used as a vein graft in a human being.

The mechanical properties of the tissue-engineered vasculature of the present invention are of major importance when determining development or appropriateness of the vessels. In particular, properties such as collagen content, cell proliferation, cell density, reactivity, and vessel constriction determine how the vessels function physically in terms of compliance and strength. It is desirable that the tissue-engineered vascular vessels of the present invention demonstrate a remarkable development in both compliance and strength in just 2 weeks.

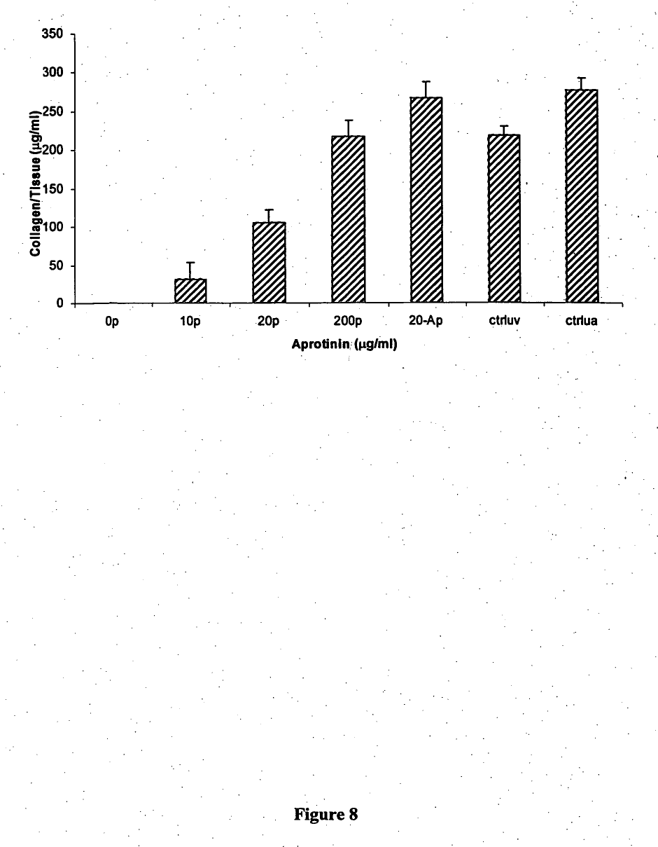

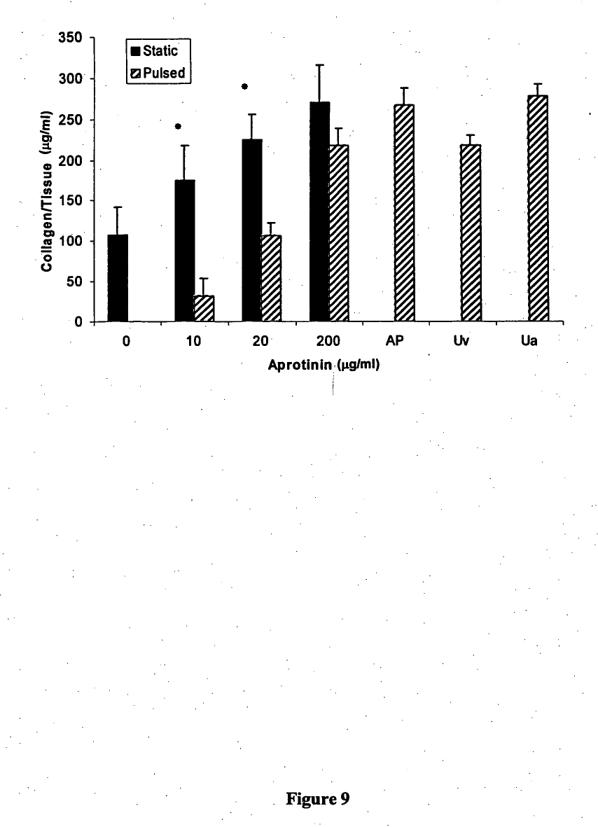

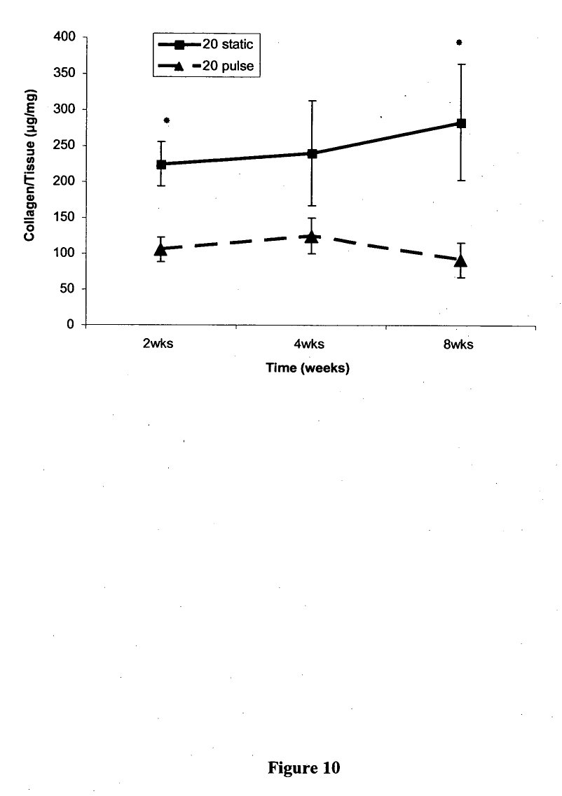

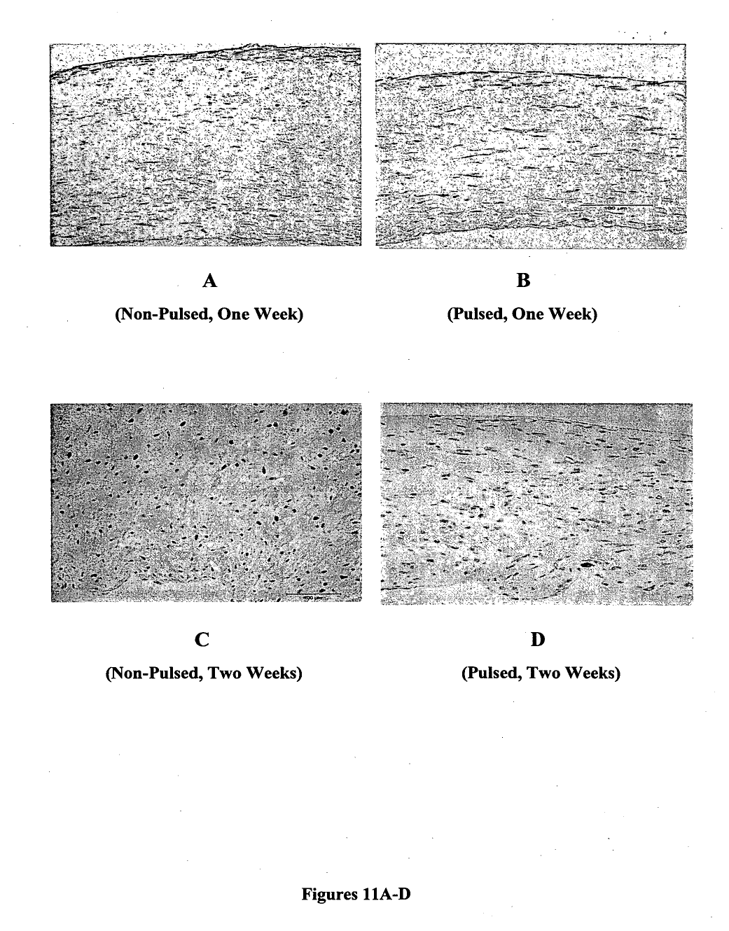

Collagen content of the tissue-engineered vascular vessels can be determined by use of the hydroxyproline assay. Using this assay, it has been shown that in the non-pulsed (FIG. 8) as well as the pulsed (FIG. 9) vessels, there is an increase in collagen content with increasing concentrations of aprotinin. The non-pulsed vessels are significantly higher in collagen content than the pulsed vessels at all concentrations of aprotinin (FIG. 10). FIG. 10 also shows that the altered pulsation vessels are greater in collagen content than both vessel groups at 20 μg/ml aprotinin, as well as being comparable to native umbilical arteries and umbilical veins. Thus, the inhibition of fibrinolysis has a stimulatory effect on the secretion of extracellular matrix. Furthermore, the addition of sufficient aprotinin can produce a tissue that is comparable to native tissue with continuous pulsation being less stimulatory than no pulsation. Results also show that there is little to no change in collagen content of the vessel constructs after 2 weeks culture time (FIGS. 11A-D). These results are supported by others who found hydroxyproline content to increase with increasing amounts of aprotinin in fibrin gels cultured in 6-well plates for 4 weeks (Ye et al., European Journal of Cardio-Thoracic Surgery 17(5):587-91 (2000), which is hereby incorporated by reference in its entirety). Border fixation of fibrin gels in culture plates has also been shown to increase hydroxyproline content (Jockenhoevel et al., European Journal of Cardio-Thoracic Surgery 19(4):424-30 (2001), which is hereby incorporated by reference in its entirety). It has been shown that other factors such as TGFP, insulin, plasmin, and time are also contributors to increasing collagen content in fibrin gels (Neidert et al., Biomaterials 23(17):3717-31 (2002), which is hereby incorporated by reference in its entirety).

The methods of producing a tissue-engineered vascular vessel are suitable for developing a vascular vessel for a particular patient. Preferably, fibrinogen and cells suitable for forming a vascular vessel are autologous, i.e., derived from the patient. More preferably, fibrinogen is isolated from the patient's blood. The fibrinogen, thrombin, and cells are then molded into a fibrin gel and incubated in a medium suitable for growth of the cells under conditions effective to produce a tissue-engineered vascular vessel. The tissue-engineered vascular vessel is then grafted into the patient from whom the fibrinogen, thrombin, and cells were isolated.

### EXAMPLES

The following examples are provided to illustrate embodiments of the present invention but are by no means intended to limit its scope.

### Example 1

### Tissue Collection

Umbilical vessels of near term fetal lambs (136 days) were collected by ligation of the distal and proximal ends with umbilical tape. The cords were allowed to drain of excess blood and the cut ends were left open to the solution. The cords were then placed in ice-cold, sterile, pH 7.4, PBS (Gibco).

### Example 2

### Cell Culture and Isolation

Ovine vascular smooth muscle cells (“OVSMC”) were isolated from umbilical vein vessels of near-term fetal lambs via explant. The vessels were collected and placed in cold PBS, with the excess connective tissue and adventitia being removed. The vessel was cut longitudinally and endothelial cells were vigorously scraped from the luminal surface and rinsed in PBS. The vessel was then cut into pieces (˜1 mm) and placed into a T-25 flask with 3 ml of medium. Cells were incubated in M199 medium supplemented with 10% fetal bovine serum (FBS), penicillin 100 U/ml, streptomycin 100 μg/ml, and 15 mM HEPES (all Gibco). Cells were used for study at passage 5 or less.

Endothelial cells were isolated from the same vessels prior to OVSMC isolation. Vessels were rinsed with PBS gently to remove any blood and debris. The vessels were then cut longitudinally and placed lumen side up. With a scalpel blade, the endothelial cells were scraped from the luminal surface with a single pass, the removed cells were then vigorously pipetted up and down in 1 ml of PBS and placed directly into a T25 flask with 4 ml of medium M199 supplemented with 15 mM HEPES, 100 U/ml streptomycin, 100 μg/ml penicillin, 1% L-glutamine, and 20% FBS. Smooth muscle cells and endothelial cells were both incubated with humidified 5% CO₂ at 37° C. Cells were passed at near confluence with 0.5% trypsin/EDTA solution. Culture medium for the vessel constructs was additionally supplemented with 50 μg/ml ascorbic acid.

### Example 3

### Fibrin Gel Preparation

Ovine fibrinogen (Sigma) was weighed at four times the final concentration (14 mg/ml; 3.5 mg/ml final concentration). This was added to 1× PBS representing one half of the total volume (1.5 ml/construct total volume). The mixture was placed in a 15 ml tube and placed on a rotation device for gentle mixing, at room temperature, about 1 hour, until all of the fibrinogen was in solution. The solution was then filter sterilized through a 0.22 μm syringe filter (Nitex), during which about half of the fibrinogen concentration was lost. The actual concentration was measured using a spectrophotometer and the concentration adjusted to 7.0 mg/ml with PBS. The fibrinogen was mixed with a thrombin fraction 1:1. The thrombin-bovine plasma origin (Sigma) was mixed in 1× PBS, 5.0 U/ml (for a final concentration of 2.5 U/ml). Calcium chloride was added to the thrombin solution at 0.55 mM. The thrombin fraction was then filter sterilized through a 0.22 μm syringe filter. The fibrinogen and thrombin fractions were not mixed until time of molding. Gelation occurred quickly; in about 2-4 seconds.

### Example 4

### Vessel Molding

The OVSMCs (3.32×10⁶ cells/ml) were added to the thrombin fraction which was mixed 1:1 with the fibrinogen fraction, resulting in a final cell concentration of 1.66×10⁶ cells/ml. The final concentration was 2.5 mM CaCl₂, 2.5 units thrombin, and 3.5 mg/ml ovine fibrinogen (all Sigma). The gel (1.5 ml/tube) was poured into a mold (3 ml syringe barrel) surrounding a 4.0 mm O.D. silastic tube prior to gelation. Gelation occurred within 2-4 seconds of mixing. It was important to mix quickly and minimally to prevent gelation from occurring prior to molding. The two fraction mixing method allowed for a uniform distribution of cells within the gel as it polymerized within seconds of mixing. The ability to obtain a homogenous cell seeding contributed to an increase of extracellular matrix secretion. The mold was then placed in the incubator for 30 minutes. After incubation, the mold was removed and the fibrin tube was placed into culture medium (30 ml).

### Example 5

### Incubation of Tissue-Engineered Vessel Constructs

Vessel constructs were left on 4.0 mm silastic tubing in which they were molded, and placed in a 50 ml conical with 30 ml of culture medium. The caps (fixed with 0.22 μm filter) were either attached to the pulsation device or left unattached. The constructs were incubated in a CO₂ incubator at 37° C. and 5% CO₂. Forty-eight hours after vessel molding, aprotinin (0-200 μg/ml) was added. Some of the vessel constructs were connected to a pneumatic pulsation system, representing a 5-10% radial distention at 60 beats/minute, and one of two pulsation time interval schemes (continuous or 1 hour per 12 hours).

### Example 6

### Aprotinin

Aprotinin (Sigma), a competitive serine protease inhibitor which forms stable complexes with and blocks the active site of enzymes, was added to the fibrin mixture at 0, 10, 20, or 200 μg/ml of vessel medium. Aprotinin was reconstituted using culture medium, filter sterilized through a 0.22 μm syringe filter, and stored at 2.0 mg/ml/vial at 2-8° C.

### Example 7

### Pulsation

Some of the vessel constructs were placed on a pneumatic pulsation device. This device was connected to an air source that provided 60 PSI to the inlet line which passed through a solenoid valve. This solenoid valve was controlled by a 60 cycle timer. The electrical outlet of the timer was controlled by a 24 hour clock with preset 30 minute intervals (used for the altered pulsation group; 1 hour/12 hours). The air then passed through a line into the incubator which connected to any number of vessel constructs arranged in a series configuration. The silastic tubing of each construct was sealed at the distal end. The pulsator was 0.5 seconds on and 0.5 seconds off for each pulsation. A pop-off valve was set to control the maximal pressure of the system which produced a 5-10% radial distention of the silastic tubing. This was measured with a digital micrometer. The pulsation wave was recorded using a Gould pressure transducer (P53), a Gould recorder system, and a BioPac A/D converter software system interfaced with an IBM computer. The pulsation scheme was maintained and monitored for each pulsation group.

### Example 8

### Isolated Tissue Bath Study

The tissue-engineered vessel constructs were removed from the silastic tubing mandrel, cut circumferentially in widths of 2-3 mm, and placed into the isolated tissue bath in a standard Krebs-Ringer solution. The constructs were continuously bubbled with 94% O₂ and 6% CO₂ to obtain a pH of 7.4, a Pco₂ of 38 mmHg, and a Po₂>500 mm Hg. The temperature was kept at 37° C. The Krebs-Ringer solution consisted of: in mmol/L; NaCl 118, KCl 4.7, CaCl₂ 2.5, KH₂PO₄ 1.2, MgSO₄ 1.2, NaHCO₃ 25.5, glucose 5.6. The tissues were placed into the system by inserting two stainless steel hooks into the lumen. Mechanical activity was recorded isometrically by a force transducer (Statham UC 2) connected to one of the steel hooks. The vessels were then equilibrated for 30-60 minutes before a passive tension of 1.0 gram was applied. Over the next 60 minutes, the constructs were rinsed 3 times and the tissue tension readjusted to 1.0 gram at a stable stretched length. Pharmacological agents were added to the bath to elucidate vessel construct function. Constrictions were elicited by adding 118 mM KCl for 15 minutes or until tension was stable. Dose response to norepinephrine at 10⁻⁸ to 10⁻⁶ mol/L, and U46619 (Thromboxane mimetic) at 10⁻⁶ mol/L was determined. Relaxations were elicited by dose response curves to norepinphrine (10⁻⁶) and U46619 (10⁻⁶) constriction by a sodium nitroprusside derivative (SNAP) 10⁻⁸ to 10⁻⁶ mol/L and isoproterenol 10⁻⁸ to 10⁻⁶ mol/L.

### Example 9

### Histology

After removal of the vessel constructs from the silastic tubing, sections were removed for histological examination. The sections were immediately placed into 10% buffered formalin (Fischer Scientific). The sections were left overnight and then washed in tap water for one hour prior to a series of dehydration steps and embedding in paraffin. The paraffin blocks were cut 4 μm in thickness in preparation for various specific immunostaining.

### Example 10

### Hemotoxylin and Eosin

Sections of the vessel constructs were deparaffinised and rehydrated to distilled water. Slides were placed into hematoxylin (Harris, Sigma) for 1 minute, then washed under running tap water for 5 minutes. Slides were then placed into eosin y solution for 3 minutes. Sections were then dehydrated through a series of ethanols and xylene before being cover slipped using mounting media (Permount, Sigma).

### Example 11

### Mason's Trichrome Stain

Vessel construct sections were deparaffinised and rehydrated to distilled water. Slides were placed into Mason's Trichrome Stain for 1 minute, then washed under running tap water for 5 minutes. Sections were then dehydrated through a series of ethanols and xylene before being cover slipped using mounting media (Permount, Sigma).

### Example 12

### Proliferating Cell Nuclear Antigen (PCNA) Stain

Proliferating cell nuclear antigen stain (“PCNA”) was used to identify and quantitate the percentage of proliferating cells within the tissue-engineered vessel constructs of the present invention. PCNA is a 36 kD molecule highly conserved between species. PCNA functions as a co-factor for DNA polymerase delta in S phase and also during DNA synthesis associated with repair. The PCNA molecule has a half life greater than 20 hours and, therefore, may detect non-proliferating cells in Go phase. Tissue section were fixed in 10% buffered formalin, and paraffin embedded. Tissue sections were cut at 4 μm and placed on positive slides. Sections were then deparaffinised and rehydrated to distilled water. Endogenous peroxidase was blocked by placing sections in 0.55 hydrogen peroxide/methanol for 10 minutes and then washed in tap water. Antigen retrieval methods were then applied. Sections were either boiled in 0.01M citrate buffer, or placed in a microwave for 30 seconds on low power. Sections were then washed 1×5 minutes in This buffer solution (TBS). Sections were placed in diluted normal serum for 10 minutes, incubated with primary antibody, and washed in TBS 2×5 minutes. Sections were also incubated with biotinylated secondary antibody, and washed in TBS 2×5 minutes. Slides were then incubated in ABC reagent (streptavidin/peroxidase complex), and washed in TBS 2×5 minutes. Slides were incubated in DAB (peroxidase substrate), and washed thoroughly in running tap water. The slides were then counterstained with hematoxylin, dehydrated, and mounted.

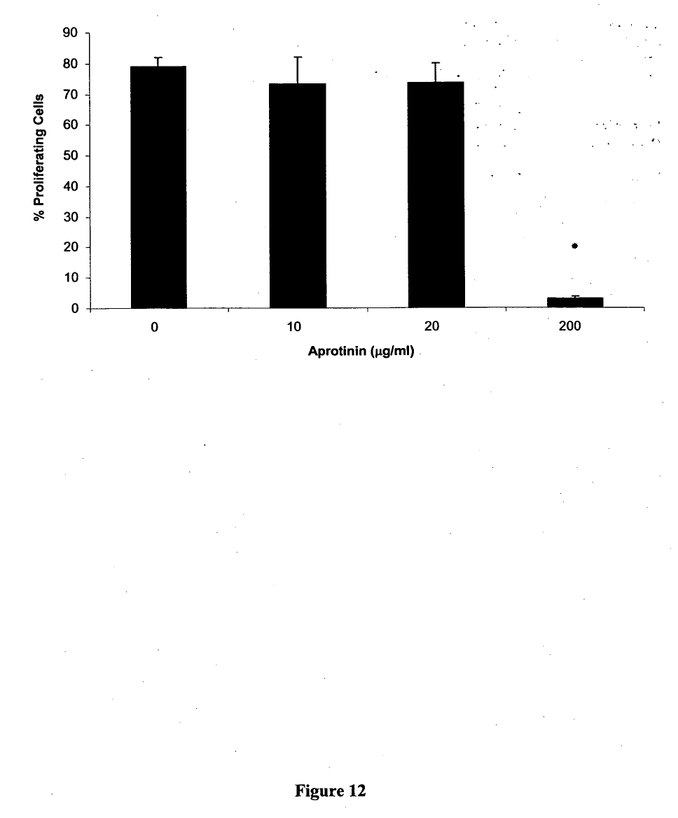

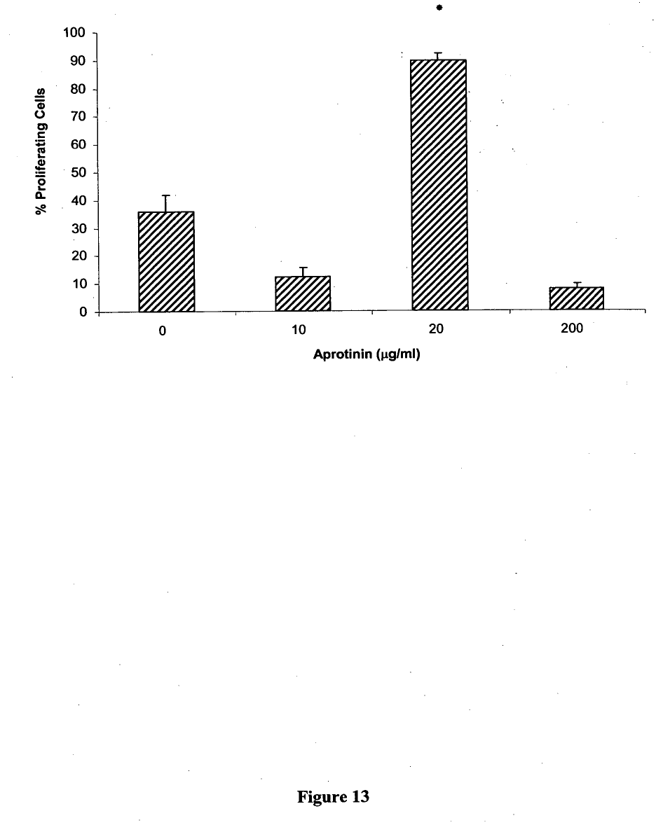

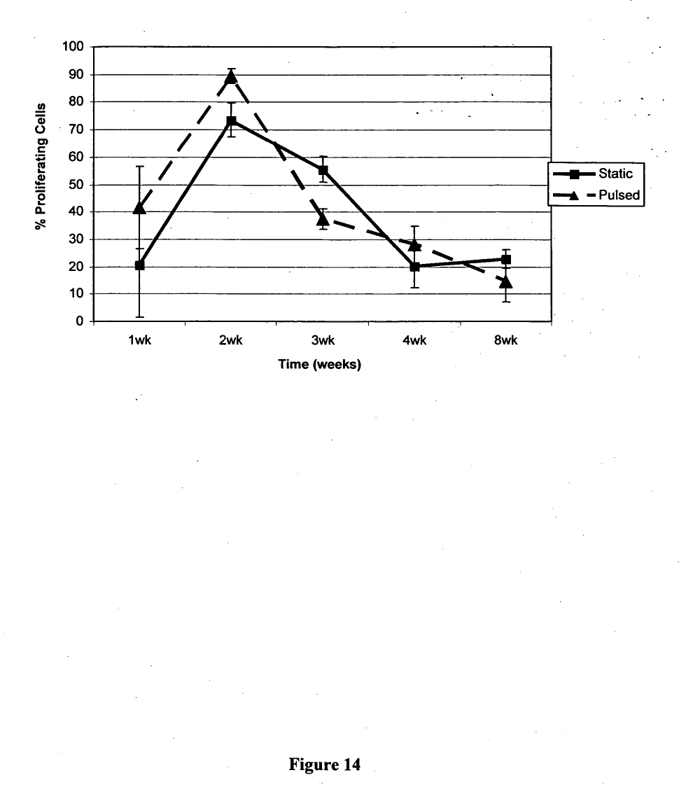

Results showed that at two weeks in the non-pulsed vessel group there was a high level of cell proliferation at lower concentrations of aprotinin. However, at 200 μg/ml aprotinin, there was a significant decline in cell proliferation (FIG. 12). This indicated that a degree of fibrinolysis was required to stimulate VSMC proliferation in fibrin gel constructs. However, in the pulsed vessels, there was a low level of cell proliferation at 0 and 10 μg/ml aprotinin with a similar level to the non-pulsed vessels at 20 and 200 μg/ml (FIG. 13). This supported the idea that a degree of fibrinolysis is required for cell proliferation and also that too much can be inhibitory. Apparently, the pulsed vessels have a higher rate of degradation, due to upregulation of secreted proteases, than the non-pulsed vessels, so that at lower levels of aprotinin and pulsation there was greater degradation. Increased degradation may inhibit cell proliferation due to loss of cell contact and adhesion with the extracellular matrix. In a study of cell proliferation over an 8 week time period using 20 μg/ml of aprotinin in the non-pulsed and pulsed vessel groups, it was demonstrated that the two groups were very similar over time but had a maximum proliferation at the 2 week time point (FIG. 14). These results indicated that 20 μg/ml aprotinin is an optimal concentration to support cell proliferation, and that at two weeks the VSMCs are exhibiting a maximal synthetic phenotype.

### Example 13

### Cell Density

Histological sections of the vessel constructs that were previously prepared with H&E Stain were used to count total number of cells per area. Random high powered fields were measured using Photo Spot software to calculate the actual surface area. The hematoxylin nuclear stain was used to identify the number of cells per high powered field. This was then used to calculate the number cells per surface area (cells/mm²).

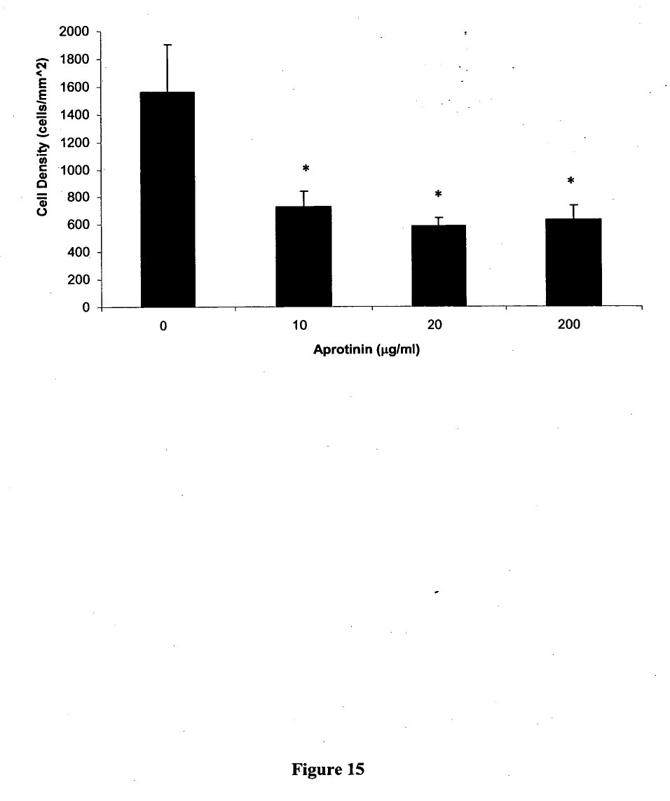

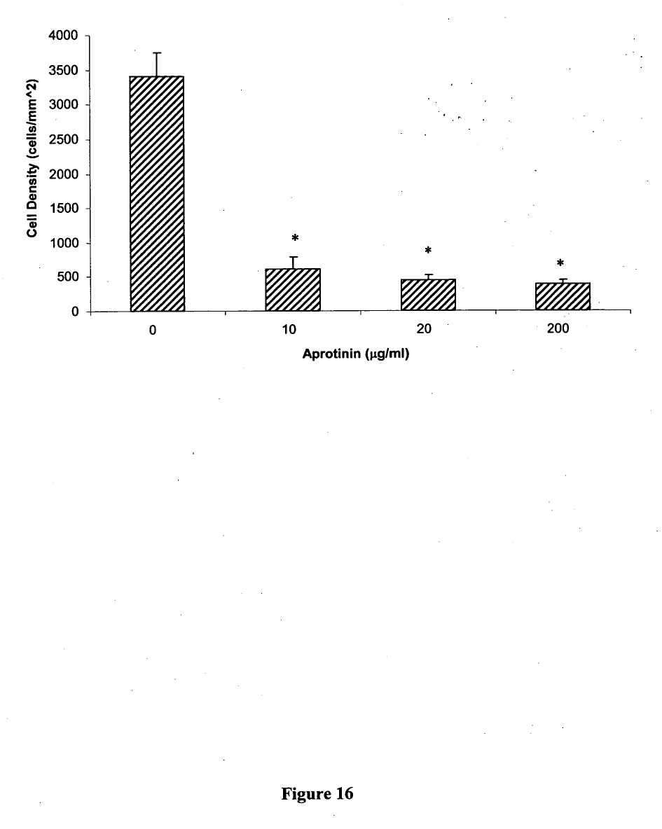

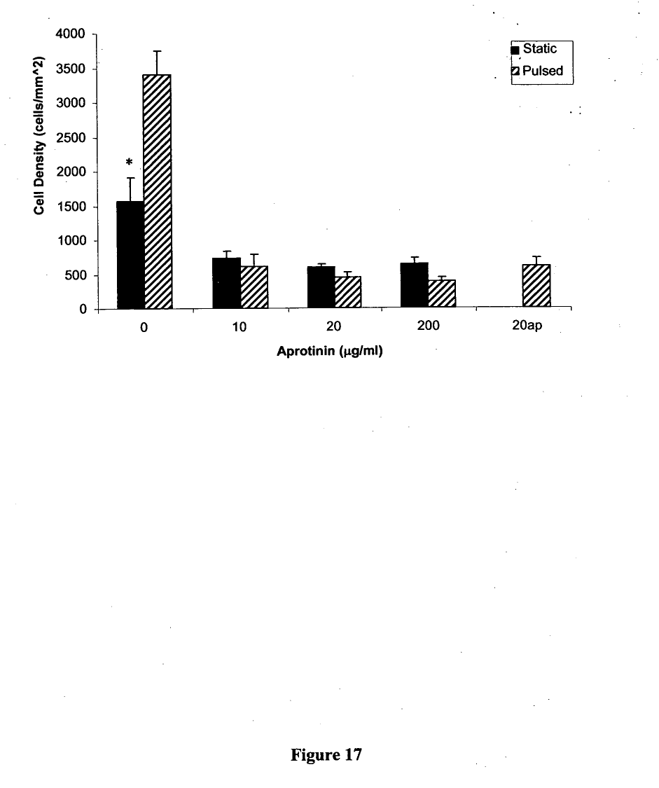

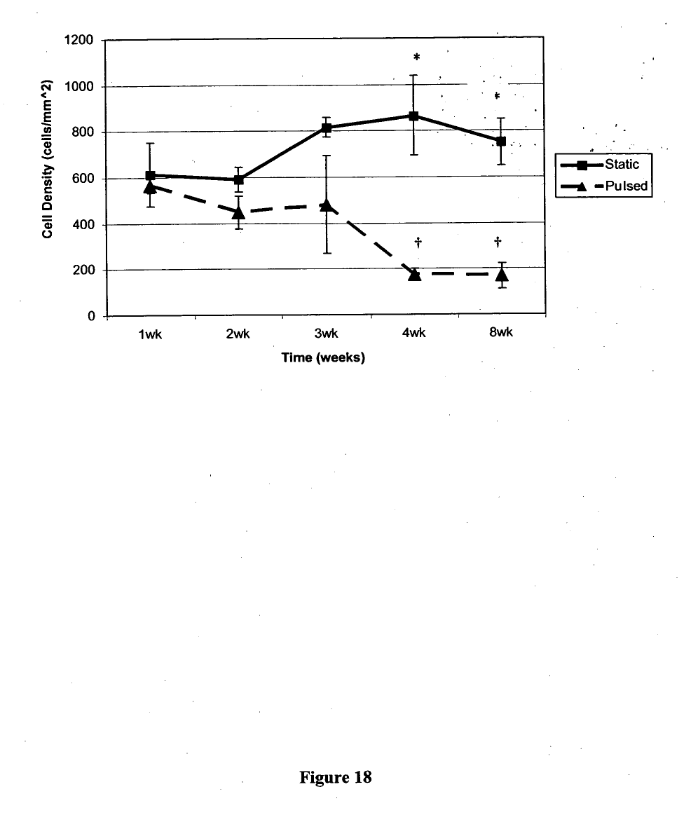

Results showed a significant trend for both vessel groups to decrease cell density with increasing amounts of aprotinin (FIGS. 15 and 16). Cell density was significantly higher at 0 μg/ml aprotinin versus a much reduced cell density at 10, 20, and 200 μg/ml aprotinin. Thus, there was a high degree of fibrinolysis occurring at 0 μg/ml aprotinin, causing the cells to concentrate due to the loss of matrix. This difference was greater in the pulsed vessel group than the non-pulsed (FIG. 17). When the trend was examined over time using 20 μg/ml aprotinin, there was a divergence in cell density from 3 weeks to 8 weeks, with significant differences at 4 and 8 weeks (FIG. 18). This divergence at later times indicated a change in balance occurring between synthesis and degradation of the matrix or development in the non-pulsed group and possibly cell death or cell loss in the pulsed group. It has previously been noted that cell proliferation at the later time points was relatively low and stable, thus ruling out a significant decrease in cell proliferation as being responsible.

### Example 14

### Tissue Weights

Tissue sections were weighed and some were placed in 10% buffered formalin for paraffin embedding, while others were placed in isolated tissue baths. The weights of the sections were added to obtain total construct weight.

### Example 15

### Hydroxyproline Assay

Native tissue and vessel constructs were assayed for hydroxyproline using a modification of the methods of Reddy and Enwemeka (Clinical Biochemistry 29(3):225-9 (1996), which is hereby incorporated by reference in its entirety) to determine total collagen content. Tissues were first dabbed dry, weighed, transferred to eppendorf tubes, and then lyophilized. Samples were mixed with 2N sodium hydroxide in a total volume of 50 μl and hydrolyzed by autoclaving at 120° C. for 20 min. To the hydrolyzate was added 450 μl of chloramines-T solution containing 1.27 gm chloramines-T (Sigma) dissolved in 20 ml 50% n-propanol (Fisher) and brought to 100 ml with acetate citrate buffer containing 120 gm sodium acetate trihydrate (Fisher), 46 gm citric acid (Fisher), 12 ml acetic acid (Fisher), 34 gm sodium hydroxide bringing to 1 liter with distilled water and pH to 6.5. Mixing gently, the oxidation was allowed to proceed for 25 minutes at room temp. Samples were then gently mixed with 500 μl of Ehrlich's Reagent containing 15 gm p-dimethylaminobenzaldehyde (p-DMBA) (Sigma) dissolved in n-propanol/perchloric acid (2:1 v/v) (Fisher) and brought to 100 ml. The resulting 96-well plate of 200 μl samples was read using a spectrophotometer set to 550 nm to determine optical density, which was then correlated with collagen amount using a standard curve and a conversion factor of 8.0 μg collagen to 1 μg 4-hydroxyproline (Edwards and O'Brien, Clinica Chimica Acta 104(2):161-7 (1980), which is hereby incorporated by reference in its entirety).

### Example 16

### Stretch, Break, and Length Measurements

Stretch, break, and length measurements were taken at the time the tissues were mounted in the isolated tissue bath. Following the reactivity studies in the isolated tissue bath, the stretch length of the tissue was measured with a micrometer from hook to hook, representing ½ of the perimeter. At this time, 1 gram of force had been applied to the hooks holding the vessel construct. This length was correlated with a numerical value on the micromanipulator associated with the force transducer and upper hook. From this point only the micromanipulator was read for adjusted length measurements. The length-tension curve was collected by incrementally increasing the force applied by turning the micromanipulator and reading the micromanipulator for new adjusted tissue stretch length. This procedure was done at predetermined increments until breakage occurred. It was at this point that a final stretch length was read and the maximal applied force was calculated.

### Example 17

### Implantation of Vessel Grafts

All procedures and protocols in this study were approved by the Laboratory Animal Care Committee at the State University of New York at Buffalo. Dorset cross castrate males 10 to 12 months of age (−25 kg) were fasted 24 hours prior to surgery. Anesthesia was induced with sodium pentathol (50 mg/animal) and maintained with 1.5-2.0% isoflourane through a 6.0 mm endotracheal tube using a positive pressure ventilator and 100% oxygen. The left external jugular vein was exposed through a longitudinal 8 cm incision. Following isolation of the vessel and tying small collateral vessels, 3000 units of heparin sulfate were administered prior to clamping the proximal and distal ends of the graft site. The vessel construct was inverted placing the endothelium to the luminal side of the graft. The external jugular was transected and a 1.0-1.5 cm segment of the vessel was sutured into place using continuous 8-0 proline cardiovascular double armed monofilament suture (Ethicon). Vascular clamp was slowly removed and flow was resumed through the vessel graft. A radiopaque tie was loosely secured at the distal end of the vessel graft as a marker of placement. The incision was closed using 2-0 vicryl in layers (facia, subcutaneous skin). The animal was recovered and monitored daily for adverse affects; angiograms were performed at 4 weeks post grafting. At the various endpoints, the animal was killed using 10 ml concentrated sodium barbiturate (Fatal Plus). The vessel graft was removed with distal and proximal native tissue left intact. Samples were taken for histological study and reactivity study.

### Example 18

### Endothelial and VSMC Isolation and Identification

Endothelial cell isolations were done using various techniques. Enzymatic isolation using collagenase was initially used. The technique was highly sensitive to collagenase concentration, temperature, time, and each preparation. This resulted in varied cell number, but mostly in contaminating cell types. Therefore, the method of scraping was used to obtain more consistent cell isolations. Through the use of DiI-Ac-LDL, a purified low density lipoprotein acetylated and labeled with the fluorescent probe DiI, endothelial cells in culture were identified and the purity was then established using flow cytometry and fluorescent microscopy. Cultures were also identified by their typical cobblestone morphology. Endothelial cells were found to be highly proliferative and maintained a uniform phenotype for multiple passages. Therefore, endothelial cells were used for experiments up to passage 12. Vascular smooth muscle cells were also initially isolated using collagenase digestion following the removal of the endothelium and adventitia. Similar results were observed—i.e., endothelial cells were often contaminants. The explant method was then employed, and the purity of the cell type was improved. The cell type was confirmed using a fluorescent marker, anti-smooth muscle myosin IgG, to label smooth muscle cells for identification using flow cytometry and fluorescent microscopy. Smooth muscle cells were also identified by cell morphology. Vascular smooth muscle cells change phenotype due to various stimuli. Therefore, smooth muscle cell cultures were used in experiments when representing a synthetic phenotype prior to passage 5.

### Example 19

### Flow System and Tissue Chambers

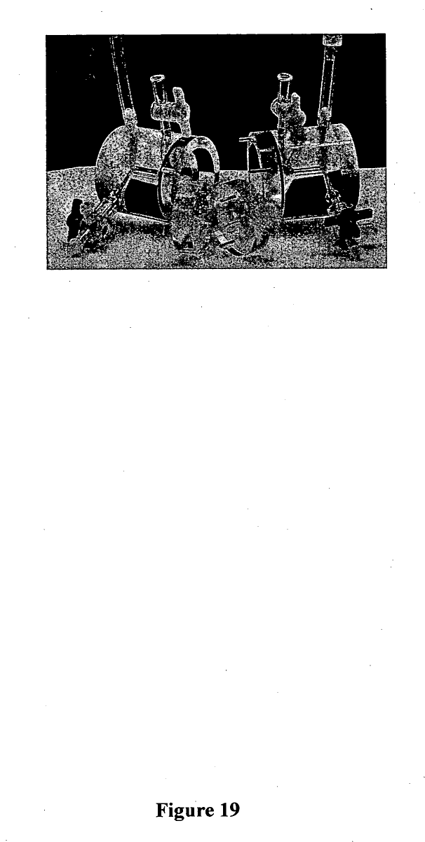

To study the development of a tissue-engineered construct, an appropriate chamber and flow system was needed. The criterion was such that a flow and/or pulsatile pressure could be applied to the construct and to have control over temperature, gas exchange, and flow conditions. A Ussing chamber was modified to achieve these conditions when placed into a flow system (FIG. 19). The temperature was controlled with a water circulating heat block. Gas exchange was controlled with a multigas flow meter exchanging gas above the media in the reservoirs, and the flow was controlled with a peristaltic roller pump with variable roller number pump heads, tubing diameters, downstream flow resistors and pump speeds. This system allowed for long term (demonstrated for 140 days) development and/or conditioning of the tissue constructs. This system was used with decellularized scaffolds and synthetic polymer scaffolds. A different system was used for the gel scaffolds that were studied. This system utilized a molding chamber, culture chamber, pneumatic pulsation device, and the ability for luminal flow control. This system was used for up to 56 days.

### Example 20

### Scaffold Materials Used for Vessel Constructs

Current methods of tissue-engineering have employed the use of various types of scaffold materials. The most abundant and readily available is that of decellularized tissue. Deepidermalized dermis was first utilized to test if vascular cell types could be seeded onto these types of scaffolds and the effectiveness of the cell seeding. Skin possesses a basement membrane for a sided differentiation and a loose type I collagen dermis on the underside. Vascular smooth muscle cells were seeded to the dermis underside and endothelial cells seeded to the basement membrane containing surface. Results indicate that there was good cell attachment to both surfaces. However, with time, there was a thickening of the endothelium and the vascular smooth muscle cells demonstrated poor infiltration and migration into the loose type I collagen dermis underside. These tissues were tested for reactivity in an isolated tissue bath sided system and demonstrated no ability to respond to various vasoactive substances. The tissues were formalin fixed and paraffin embedded. Stained with hemotoxylin/eosin and Mason's trichrome. With the dermatome available for dermal collection, the dermal matrix is about 300 to 400 μm in thickness.

A commercially available product was then used that was similar to the dermis but was 225 μm in thickness. VivoSIS™ is a porcine small intestine submucosa cell culture sheet. It also has a basement membrane associated with one surface and a loose type I collagen component on the opposite surface. This scaffold material was seeded similar to that of the decellularized dermis with endothelial cells on the basement membrane surface and smooth muscle cells on the opposite surface. Results indicated that at 7 days there was a confluent endothelium similar to the dermis scaffold but that there was a greater seeding of smooth muscle cells into the type I collagen underside. Over time (up to 140 days), there was an improved development of the scaffold that exceeded that of the decellularized dermis. However, there was still, even at 140 days, an incomplete cellularization of the collagen matrix with smooth muscle cells.

The scaffold densities of the natural materials may be too great for rapid cell infiltration, migration, and cell seeding. Based on this, a synthetic polymer was tried which possessed a porosity that could be controlled in its fabrication. There are many methods of fabricating these polymers into tissue scaffolds. It was first tried to fabricate scaffold material using 50/50 PLGA and temperature induced phase separation (TIPS). Using a controlled temperature during the quenching phase it was possible to control the pore diameter of the material. Thin slices (about 200 μm) were cut and then seeded with smooth muscle cells. Results showed that when both diameter (10-20 μm and 150-200 μm) scaffold materials were used, the smaller diameter material did not allow enough cell infiltration and seeding. When the larger pore material was used, it was too inconsistent in contiguous surface. There was an inconsistent surface available for cell seeding which resulted in gapping holes as the material degraded at a controlled rate by surface hydrolysis. A 50/50 poly lactic-glycolic acid (PLGA) fiber mesh (about 200 μm thick) was then used. This material had a high porosity and functional pore size with the fiber mesh network providing a large surface area. Smooth muscle cells were seeded into this material resulting in a poor distribution of seeded cells and non-uniform development of tissue. Following a short period of time (7-14 days), these tissues were fragile, demonstrating poor cohesiveness and integrity.

Gel scaffolds have advantages of providing a media that optimizes cell seeding, uniform distribution, controlled shape, and cellular alignment via constrained compaction. Collagen gels were used in the earliest development of tissue-engineered vascular constructs. Even though they had shown poor strength and development, it was thought that if proper stimuli were applied this process may be enhanced. Because of the several advantages of gels, smooth muscle cells were added to the thrombin fraction of a 2.0 mg/ml collagen mixture that was molded around a 4.0 mm silastic tubing. Results showed a uniform distribution of cells throughout the gel and a cellular alignment circumferential around the central mandrel. The alignment was predominantly toward the outer portion of the gel. These collagen gel constructs, when tested for vasoreactivity, demonstrated minimal ability to constrict and relax to vasoactive substances. When exposed to a pulsation of 60 beats/minute and a 5-10% distension, the constructs did not display any additional integrity.

Fibrinogen is known to increase vascular smooth muscle cell secretion of extracellular matrix and migration. Fibrin gel scaffolds constructs were formed by adding 1.66 million cells/ml (vascular smooth muscle cells) to the thrombin fraction, and upon mixing with the fibrinogen (3.5 mg/ml final concentration) fraction, molding the gel around a 4.0 mm silastic tube. Some of these fibrin gel constructs were exposed to a 5-10% radial distension and a rate of 60 beats/min. Physical appearance showed a tubular construct with a high degree of integrity (FIGS. 1A-B). The non-pulsed construct (FIG. 1B) appeared to have a thicker wall and a higher degree of longitudinal compaction as opposed to the pulsed construct (FIG. 1A), which appeared to have a thinner wall and be longer in length.

### Example 21

### Fibrin Gel Compaction and Tissue Development

Histological examination of the tissue-engineered vascular vessels at 1 week under pulsed (FIGS. 2A and 2C) and non-pulsed (FIGS. 2B and 2D) conditions, stained with hematoxylin and eosin (FIGS. 2A and 2B), and Mason's Trichrome Stain (FIGS. 2C and 2D), showed a uniform distribution of cells throughout the fibrin gel construct in both the pulsed (FIGS. 2A and 2C) and non-pulsed (FIGS. 2B and 2D) condition. There was a greater degree of vascular smooth muscle cell alignment in the pulsed condition than in the non-pulsed condition (40× magnification). The force of pulsation was perpendicular to that of the cellular alignment.

The secretion of type I collagen by the vascular smooth muscle cells under a pulsed condition was observed in histological sections at 5 (FIG. 3A), 10 (FIG. 3B), and 15 (FIG. 3C) days consecutively, by using Mason's Trichrome Stain. An increased staining for type I collagen was observed within the first 10 days (20× magnifications). This was also observed in FIGS. 2C and 2D, which showed the Mason's Trichrome Stain comparing the non-pulsed (FIG. 2D) and pulsed (FIG. 2C) constructs. Similarly, FIGS. 3A-C demonstrate an increased cellular alignment with time; preferentially in the cells toward the outer edge of the fibrin gel construct.

### Example 22

### Vessel Weights, Aprotinin Concentration, and Time of Addition

In order to optimize the use of aprotinin, which inhibits fibrinolysis of the fibrin gel, different concentrations of aprotinin (0, 10, 20, 200 μg/ml) were used at various start times (0, 24, and 48 hrs.) following vessel molding. Results showed that at 10 and 20 μg/ml of aprotinin, the later (48 hrs.) the addition of aprotinin from the time of molding, the greater the total tissue weight at a two week time point. This was found to be a similar increase in the pulsed and non-pulsed condition (50% non-pulsed, 130% pulsed).

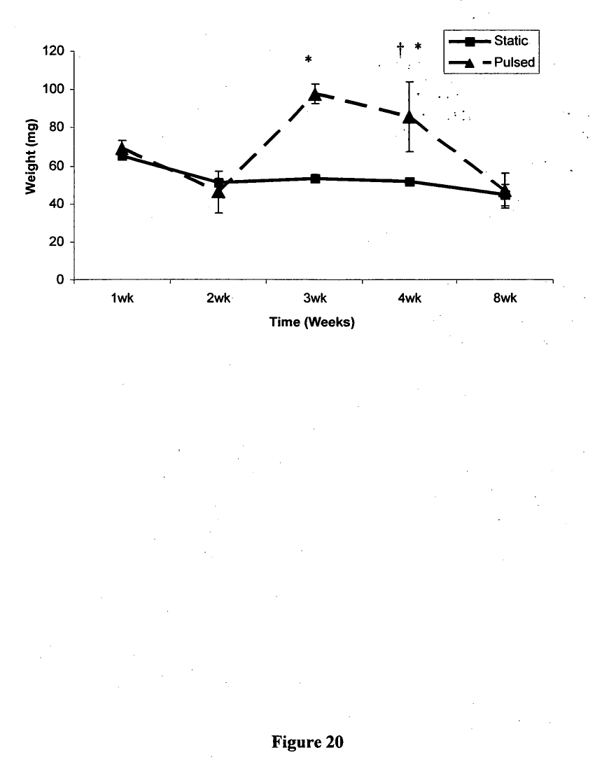

The non-pulsed fibrin gel constructs at 14 days demonstrated that higher concentrations of aprotinin increased the total weight of the constructs: 0 μg/ml, 16.6±3.1 mg, n=3; 10 μg/ml, 47.0±2.5 mg, n=4; 20 μg, 51.3±2.1 mg, n=6; 200 μg, 52.0±2.3 mg, n=4. The weight increased greatly between 0 μg/ml and 10 μg/ml aprotinin and only slightly after that (FIG. 5). The pulsed fibrin gel constructs at 14 days demonstrated higher concentrations of aprotinin (0 μg/ml, 14.6±1.4 mg, n=2; 10 μg/ml, 38.5±11.2 mg, n=4; 20 μg, 46.1±11.0 mg, n=5; 200 μg, 101.4±14.2 mg, n=5), producing an increase in total weight of the construct, with a significant increase between 0-10 μg/ml aprotinin, and 20-200 μg/ml aprotinin. Only a slight increase was observed between 10-20 μg/ml aprotinin (FIG. 6). The construct total weights were slightly lower for the pulsed constructs at 0, 10, and 20 μg/ml aprotinin, as compared to the non-pulsed constructs. However, at 200 μg/ml aprotinin, the pulsed construct weight was significantly higher than the non-pulsed. The altered pulsation group (1/12 pulsation) represents a greater total weight than both the pulsed and non-pulsed group at 20 μg/ml aprotinin (FIG. 7). Considering the change in weight of the constructs over time (1, 2, 3, 4, and 8 weeks), there was an overall slight decrease in the non-pulsed group at 20 μg/ml aprotinin (1 wk, 65.1±1.8, n=2; 2 wk, 51.3±2.1, n=6; 3 wk, 53.2±2.3, n=2; 4 wk, 51.7±1.7, n=3; 8 wk, 44.4±5.7, n=4). In the pulsed group, there was a similar overall decrease over time, with a sharp rise at 3 and 4 weeks (1 wk, 69.1±4.1, n=2; 2 wk, 46.1±11.0, n=5; 3 wk, 97.5±5.2, n=2, 4 wk, 85.7±18.2, n=3; 8 wk, 46.8±9.0, n=3) (FIG. 20). The altered pulsation group (1/12 pulsation) (2 wk, 82.1±1.3, n=5) was significantly higher than both the non-pulsed and pulsed groups at the same aprotinin concentration of 20 μg/ml.

### Example 23

### Vessel Type I Collagen Determination by Hydroxyproline Assay

Hydroxyproline is an imino acid found specifically in type I collagen at 12.5% of the total by weight. This spectrophotometric assay was used to quantitate directly the collagen content of tissue homogenates. The values are represented as μg of collagen/mg of tissue, dry weight.

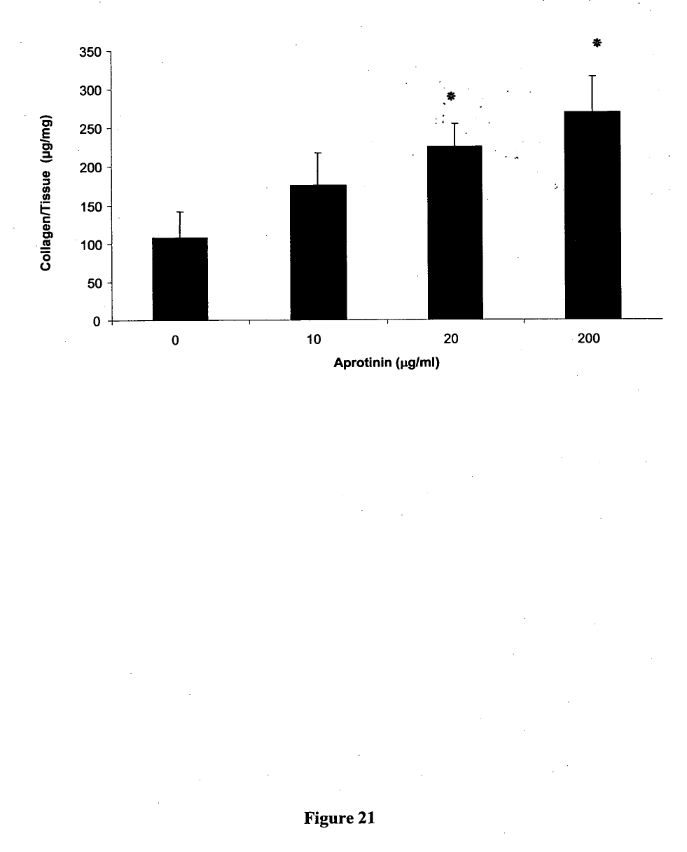

The non-pulsed fibrin gel constructs at 14 days demonstrated that higher concentrations of aprotinin resulted in a higher collagen content (0 μg/ml, 107.5±34.4 μg/mg, n=4; 10 μg/ml, 175.2±42.3 μg/mg, n=4; 20 μg, 225.0±30.2 μg/mg, n=7; 200 μg, 270.1±46.2 μg/mg, n=4). The increase was significant at 20 μg/ml and 200 μg/ml aprotinin (FIG. 21). The pulsed fibrin gel constructs at 14 days also demonstrated that higher concentrations of aprotinin (0 μg/ml, 0.0±0.0 mg, n=0; 10 μg/ml, 31.5±22.5 μg/mg, n=6; 20 μg, 105.9±16.7 μg/mg, n=6; 200 μg, 217.3±21.0 μg/mg, n=5) produced an increase in collagen content of the constructs with a significant increase at each concentration of aprotinin (FIG. 8). The construct collagen contents are lower at all aprotinin concentrations for the pulsed as compared to the non-pulsed constructs. The altered pulsation group (1/12 pulsation) (2 wks, 266.6±20.9, n=5), represented a significant increase in collagen content over the pulsed group at 20 μg/ml aprotinin and comparable to the native umbilical artery and umbilical vein (FIG. 9). Considering the change in collagen content of the constructs over time (2, 4, and 8 weeks), there was a slight insignificant increase in the non-pulsed group at 20 μg/ml aprotinin (2 wk, 225.0±30.2, n=7; 4 wk, 239.9±73.2, n=3; 8 wk, 283.5±80.3, n=4). While in the pulsed group there was a small rise at 4 wks followed by a slight decrease at 8 wks, both changes were insignificant (2 wk, 105.9±16.7, n=6; 4 wk, 124.5±25.1, n=3; 8 wk, 91.8±24.3, n=3) (FIG. 10).

### Example 24

### Cell Proliferation

Proliferating cell nuclear antigen (PCNA) was used to identify cells that were in a proliferating state using histological staining methods. FIGS. 11A-D represent constructs that were stained at 1 week (FIGS. 11A and 11B) and 2 weeks (FIGS. 11C and 11D). The constructs in FIGS. 11A and 11C were under non-pulsed conditions. The constructs in FIGS. 11B and 11D were under pulsed conditions. The PCNA antibody was visualized with diaminobenzidine (DAB) and counter stained with hematoxylin. There was little staining visualized at the one week time point as compared to the two week time point for both the non-pulsed and pulsed constructs. The non-pulsed tissue was slightly greater at both one and two weeks. The PCNA staining was quantitated by counting the total number of positive cells per high powered field and dividing by the total number of cells in the same field to obtain percent of proliferation.

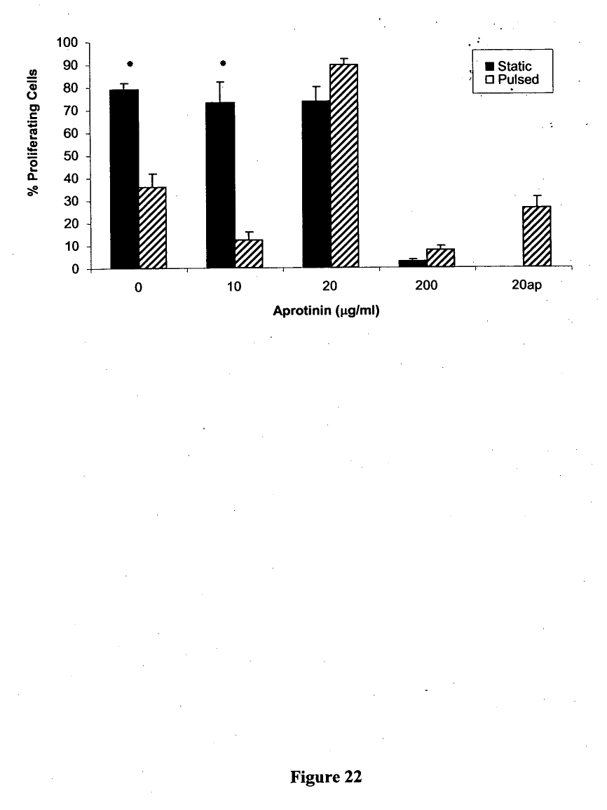

The non-pulsed fibrin gel constructs at 14 days demonstrated that higher concentrations of aprotinin resulted in no change in proliferation between 0 and 20 μg/ml. However, at 200 μg/ml, there was a significant decrease in proliferation (0 μg/ml, 79.0±3.0%, n=3; 10 μg/ml, 73.3±8.8%, n=4; 20 μg, 73.6±6.3%, n=6; 200 μg, 2.9±0.9%, n=4) (FIG. 12). The pulsed fibrin gel constructs at 14 days demonstrated a decline in proliferation at 10 μg/ml aprotinin, and then a sharp increase at 20 μg/ml aprotinin, followed by a sharper decline at 200 μg/ml aprotinin (0 μg/ml, 35.8±5.8%, n=2; 10 μg/ml, 12.3±3.6%, n=4; 20 μg, 89.6±2.6%, n=5; 200 μg, 7.7±1.9%, n=5) (FIG. 13). The cell proliferation was significantly lower for the pulsed group at 0 and 10 μg/ml aprotinin than the non-pulsed group. However, at 20 μg/ml aprotinin, both groups were equally elevated and equally depressed. The altered pulsation group (1/12 pulsation) (2 wks, 26.3±5.0, n=5) represented a significant decrease in proliferation compared to both non-pulsed and pulsed at 20 μg/ml aprotinin (FIG. 22). Considering the change in cell proliferation over time (1, 2, 3, 4, and 8 weeks), there was a significant increase in both the non-pulsed and pulsed group at 20 μg/ml aprotinin, and there was a steady decline in cell proliferation in the weeks to follow, with the non-pulsed group being equal to the pulsed group at each time point (Non-pulsed: 1 wk, 20.44±44.0%, n=2; 2 wk, 73.6±6.3%, n=6; 3 wk, 55.5±4.7%, n=2; 4 wk, 20.1±7.6%, n=4; 8 wk, 22.8±3.%, n=6; Pulsed: 1 wk, 41.7±31.1%, n=2; 2 wk, 89.6±2.6%, n=5; 3 wk, 37.5±3.6%, n=3; 4 wk, 28.0±6.9%, n=4; 8 wk, 14.7±7.5%, n=3) (FIG. 14).

### Example 25

### Cell Density within Vessel Constructs

Cell density within vessel constructs of the present invention was calculated using histology sections stained with hematoxylin and eosin. Total number of cells were counted per high powered field, divided by the area measured using Photospot Advanced software, and reported as number of cells/mm². The non-pulsed fibrin gel constructs at 14 days demonstrated a significant decrease in cell density from 0 to 10 μg/ml aprotinin and a steady cell density thereafter (0 μg/ml, 1564±340 cells/mm², n=3; 10 μg/ml, 731±108 cells/mm², n=4; 20 μg, 591±52 cells/mm², n=6; 200 μg, 635±98 cells/mm², n=4) (FIG. 15). The pulsed fibrin gel constructs at 14 days demonstrated a significant decrease in cell density from 0 to 10 μg/ml aprotinin, and a steady cell density thereafter (0 μg/ml, 3410±336 cells/mm², n=2; 10 μg/ml, 611±180 cells/mm², n=4; 20 μg, 448±71 cells/mm², n=5; 200 μg, 390±59 cells/mm², n=5) (FIG. 16). The cell density was significantly higher for the pulsed group at 0 μg/ml aprotinin than the non-pulsed group. However, at 10 μg/ml aprotinin and thereafter, both groups were decreased and equal. The altered pulsation group (1/12 pulsation) (2 wks, 608±123 cells/mm², n=5) was equal to both non-pulsed and pulsed at 20 μg/ml aprotinin (FIG. 17). Considering the change in cell density over time (1, 2, 3, 4 and 8 weeks), they were similar at weeks 1 and 2. However, by week 3, the non-pulsed group began to increase, and the pulsed group significantly begins to decrease. At weeks 4 and 8, there was a significant difference between the two groups (Non-pulsed: 1 wk, 613±140, n=2; 2 wk, 591±52, n=6; 3 wk, 817±43, n=2; 4 wk, 865±17, n=4; 8 wk, 751±101, n=6; Pulsed: 1 wk, 566±33, n=2; 2 wk, 448±71, n=5; 3 wk, 480±213, n=3; 4 wk, 175±25, n=4; 8 wk, 168±54, n=3) (FIG. 18).

### Example 26

### Reactivity of Fibrin Vessel Constructs

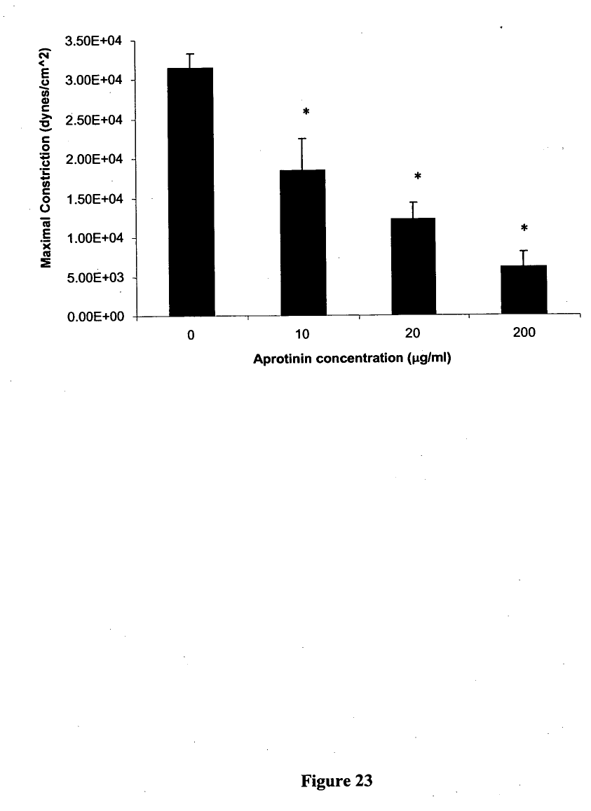

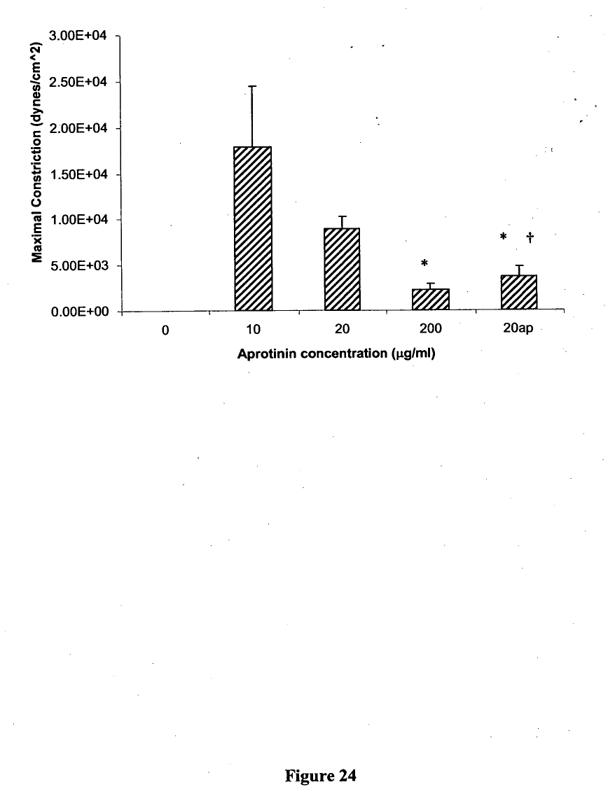

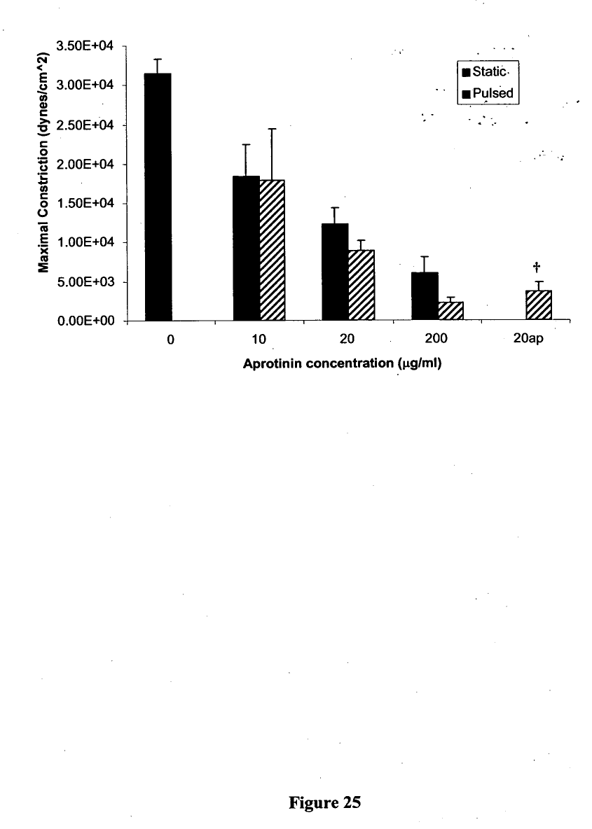

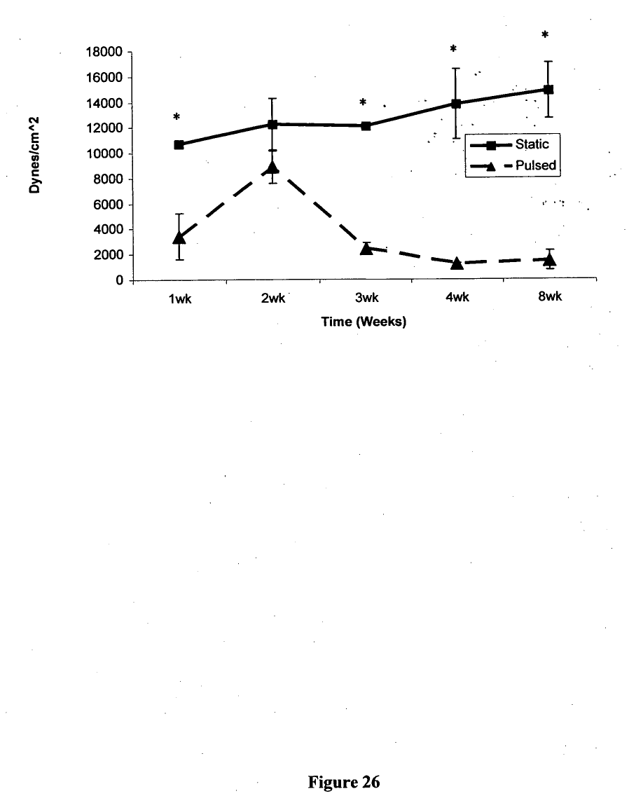

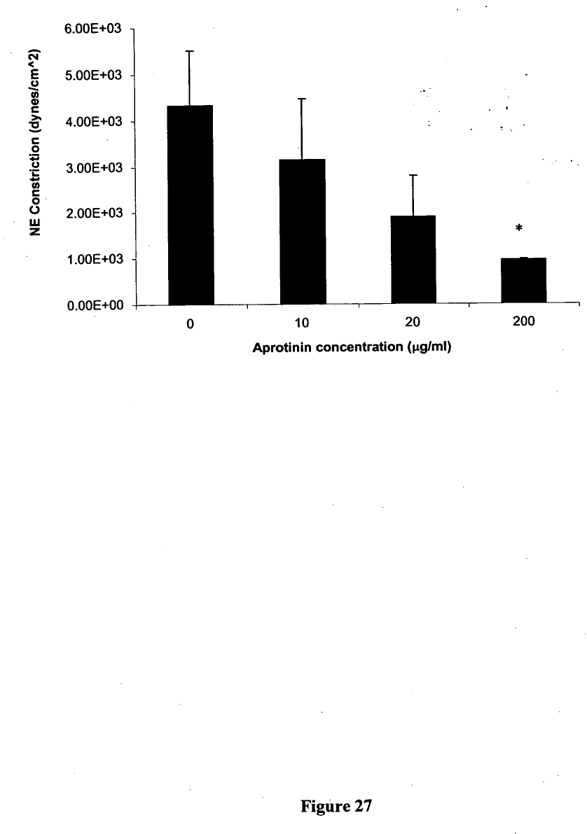

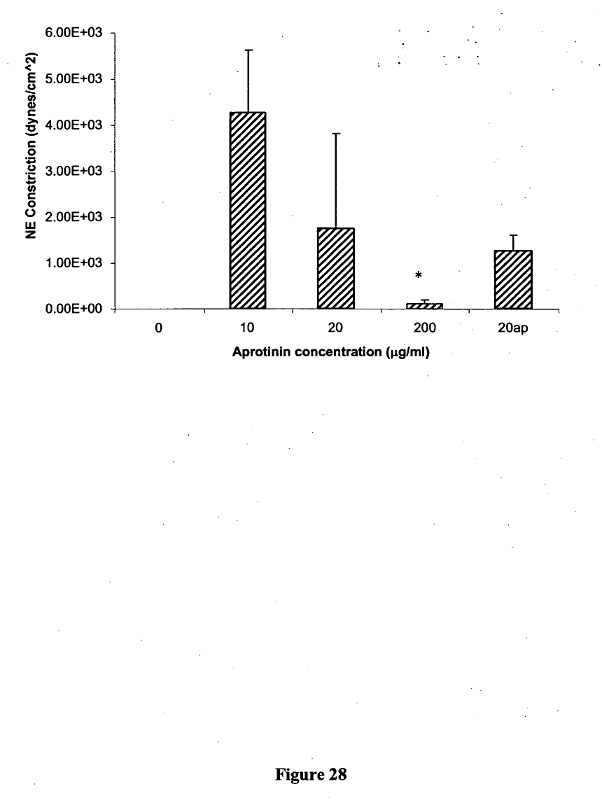

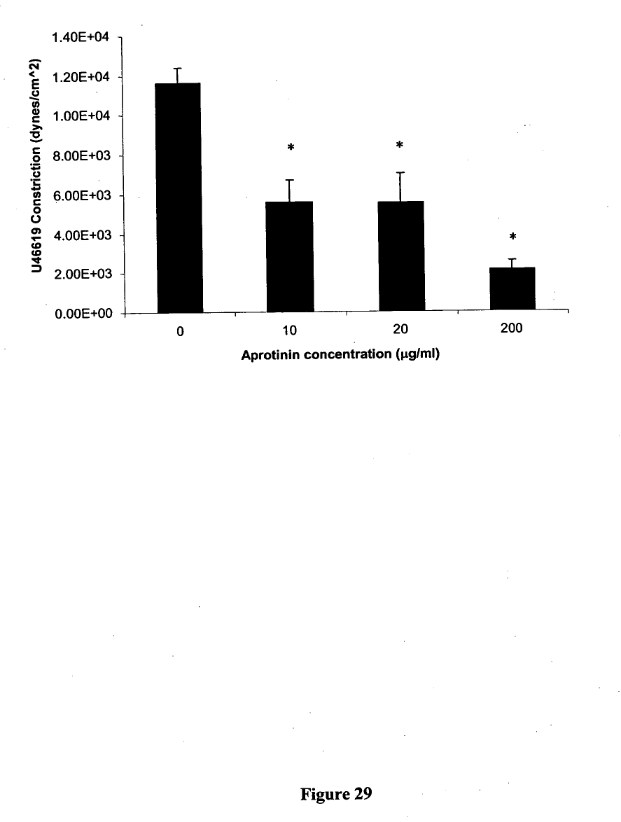

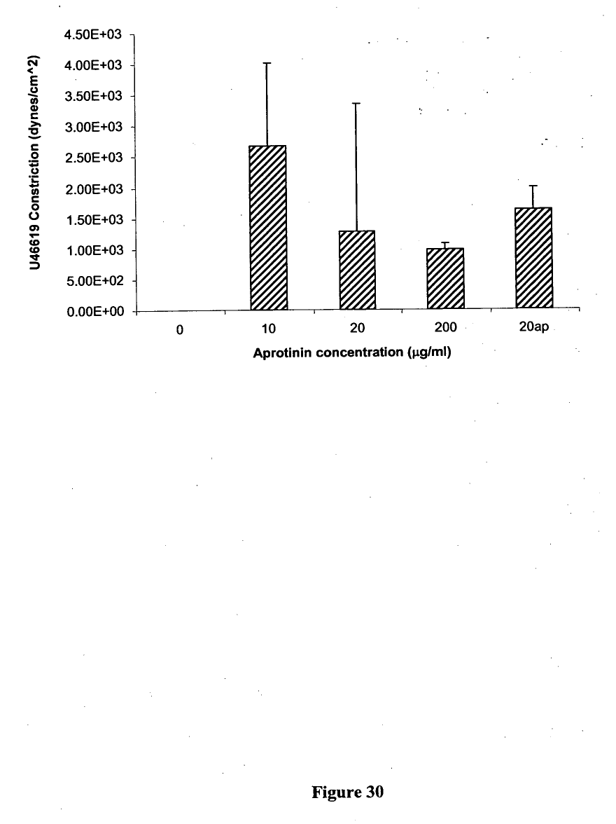

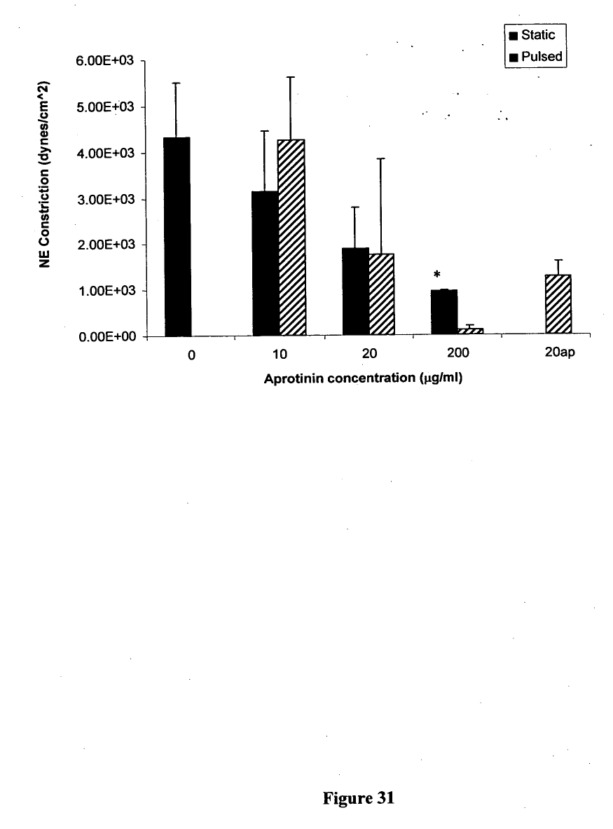

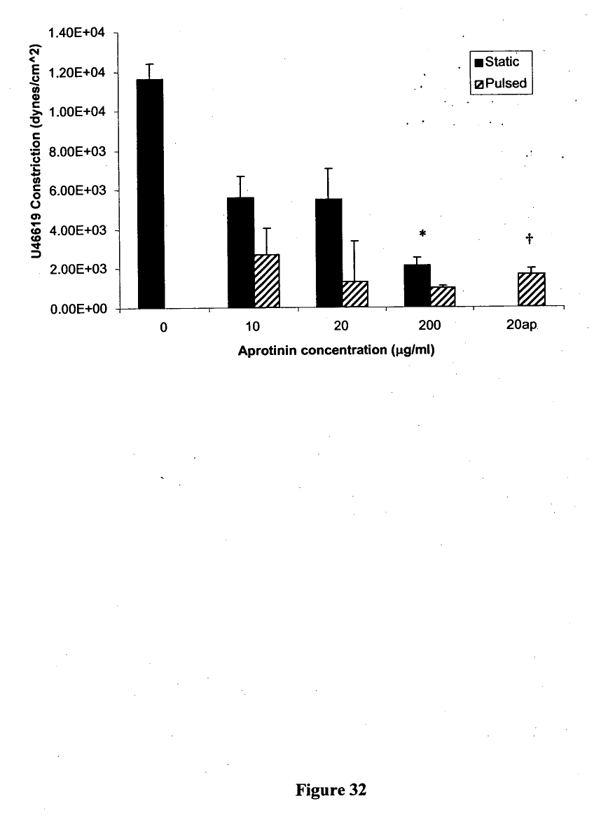

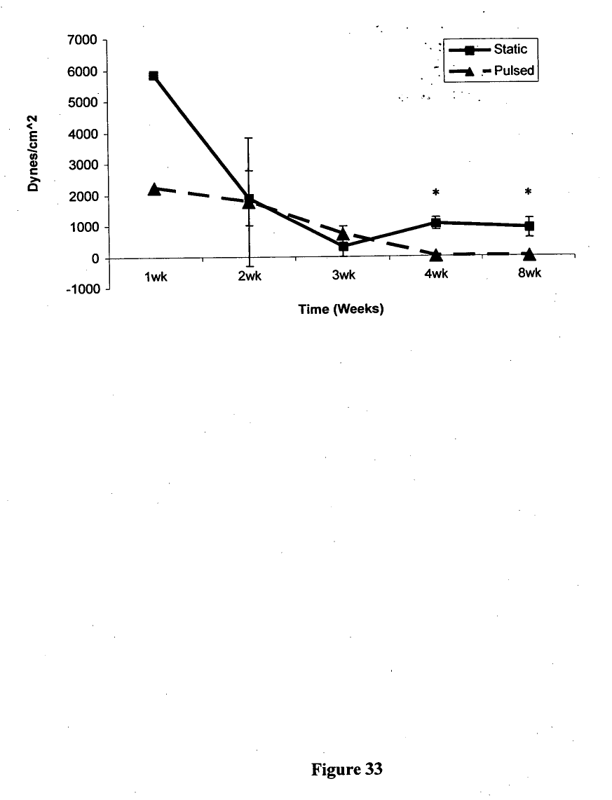

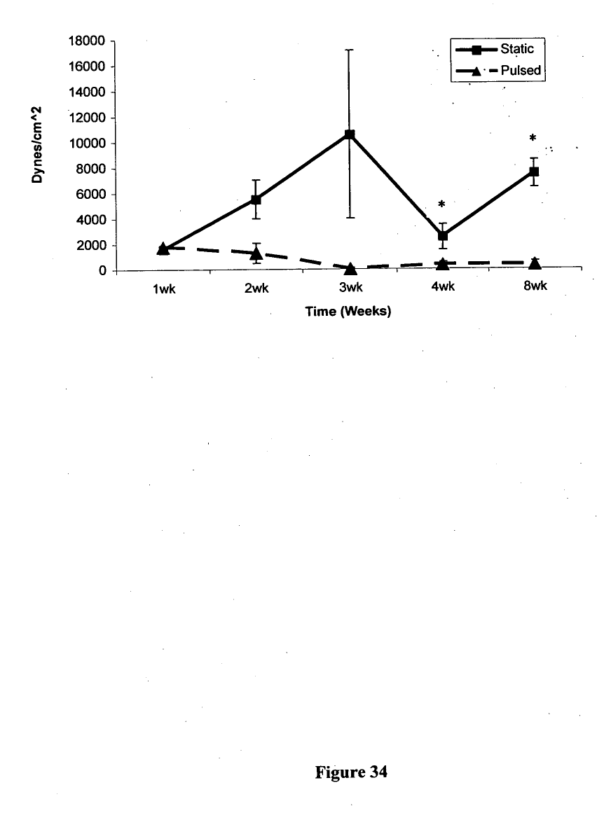

The ability of the fibrin constructs to constrict or dilate in response to vasoactive substances was measured by placing a ring of the fibrin construct into an isolated tissue bath. When exposed to 118 mM KCl, a non-receptor mediated vasoconstrictor, the constriction significantly decreased with increasing concentrations of aprotinin in both pulsed and non-pulsed constructs (FIG. 23 and FIG. 24). Non-pulsed tissues developed contractions similar to that of pulsed tissues at 10 μg/ml of aprotinin (18446±4027 dynes/cm², 19274±8302 dynes/cm²; non-pulsed and pulsed respectively) compared to a greater constriction for non-pulsed at 20 and 200 μg/ml aprotinin (12244±2083 dynes/cm², 6056±2003 dynes/cm²) than pulsed (8896±1347 dynes/cm², 2232±475 dynes/cm²) (FIG. 25). Also, over an eight week time period, the non-pulsed group constriction was considerably greater than the pulsed group (FIG. 26). Similarly, specific receptor mediated constrictors norepinephrine (3×10⁻⁶ M) and U46619 (3×10⁻⁷) (a thromboxane A₂ mimetic), demonstrated the same trend with respect to aprotinin concentration (NE, Non-pulsed: 0 μg/ml at 4743±1849 dynes/cm² to 200 μg/ml at 965±16 dynes/cm²; NE, Pulsed: 10 μg/ml at 4316±1738 dynes/cm² to 200 μg/ml at 101±102 dynes/cm²; U46619, Non-pulsed: 0 μg/ml at 1160±775 dynes/cm² to 200 μg/ml at 2140±416 dynes/cm²; U46619, Pulsed: 10 μg/ml at 2670±944 dynes/cm² to 200 μg/ml at 973±240 dynes/cm²) (FIGS. 27, 28, 29, and 30). When comparing the non-pulsed to the pulsed groups for norepinephrine and U46619 constrictions at various aprotinin concentrations, the non-pulsed group was greater than the pulsed group at all points, except for NE at 10 and 20 μg/ml aprotinin, where they were similar (FIGS. 31 and 32). Comparing the two receptor-mediated vasoconstrictor over the 8 week time period, the non-pulsed was comparable to the pulsed at 1, 2, and 3 weeks. However, at 4 and 8 weeks, the non-pulsed group was greater (FIG. 33 and 34).

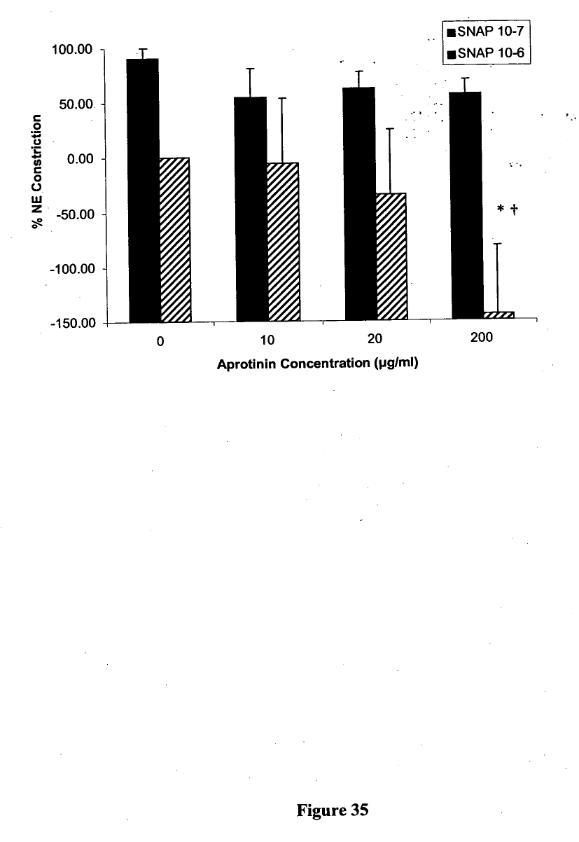

Both pulsed and non-pulsed vessel constructs constricted by norepinephrine (3×10⁻⁶) relaxed to SNAP (10⁻⁵ M), a non-receptor mediated nitric oxide donor, fully and 42% of constriction respectively. When comparing SNAP relaxations at 10⁻⁷ and 10⁻⁶ M to a norepinephrine constriction (10⁻⁶ M), they were comparable at 0, 10, and 20 μg/ml aprotinin. However, at 200 μg/ml aprotinin, the relaxation was much greater (FIG. 35). Relaxations to isoproterenol (β receptor agonist) were measured with results being much less than that of SNAP.

### Example 27

### Stretch Length at 1 Gram of Tension

Vessel constructs were mounted into the isolated tissue baths and a basal tone was applied to the construct. Native vascular tissues typically have a degree of basal tone at all times which also allows the tissue to respond either as a constriction or a relaxation in response to vasoactive stimuli. The vessel constructs were molded onto a 4.0 mm silastic tube, giving them all the same initial effective starting diameter. When 1 gram of tension was applied to all the constructs, the resulting stretch length represented a degree of elasticity at the constructs' optimal basal tone. This elasticity was compared between various culture conditions and aprotinin concentrations.

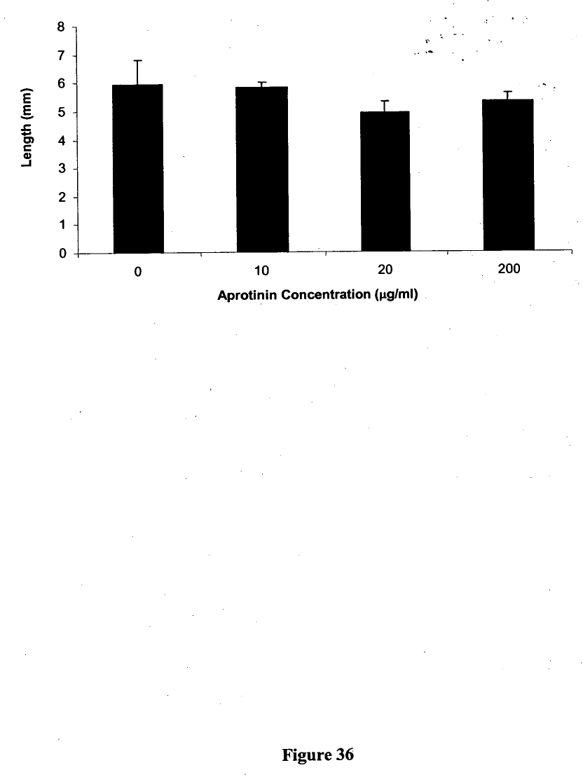

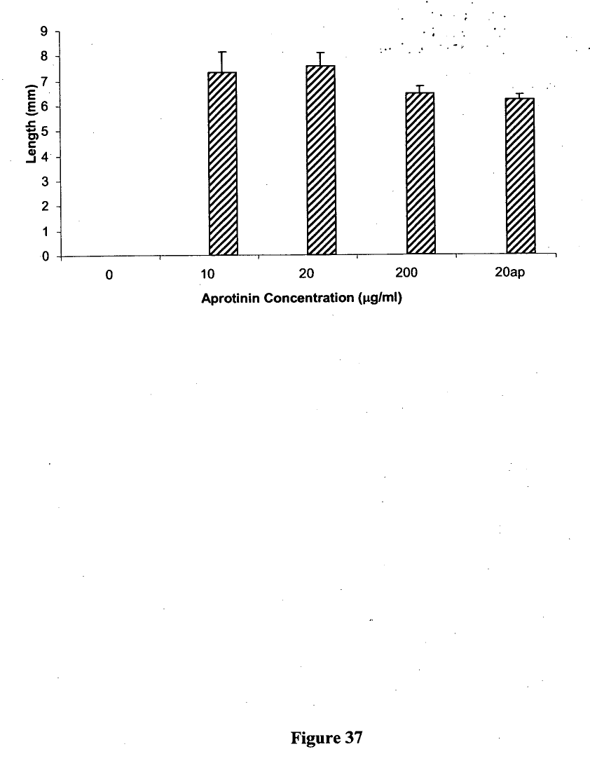

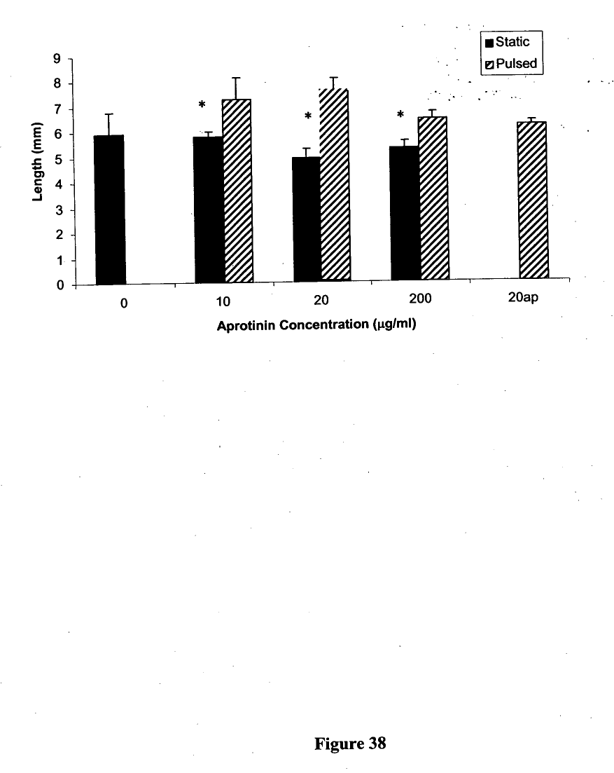

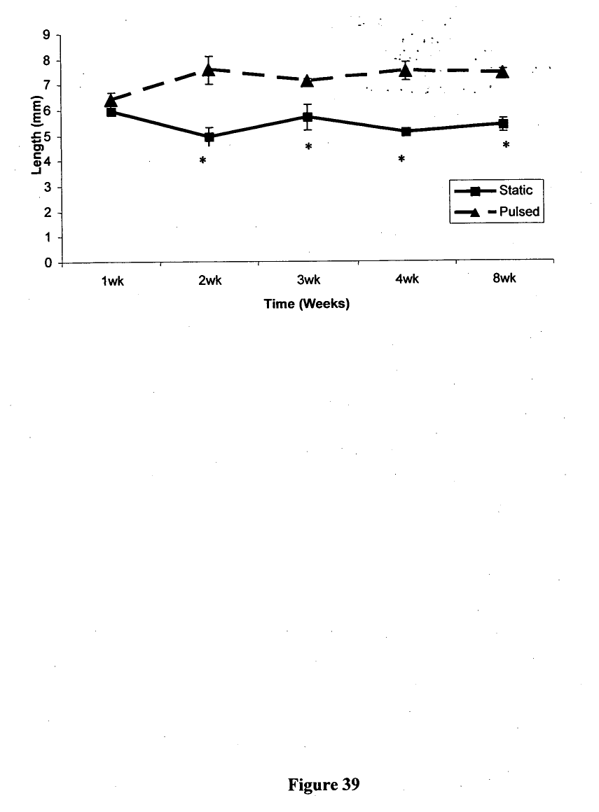

The non-pulsed fibrin gel constructs at 14 days demonstrated that higher concentrations of aprotinin resulted in a small decrease in the starting stretch length (0 μg/ml, 5.93±0.87 mm, n=3; 10 μg/ml, 5.83±0.19 mm, n=4; 20 μg, 4.94±0.38 μg/mg, n=6; 200 μg, 5.33±0.28 mm, n=4). The decrease in stretch length was not significant at any concentration of aprotinin (FIG. 36). The pulsed fibrin gel constructs at 14 days also demonstrated that higher concentrations of aprotinin (0 μg/ml, 0.0±0.0 mg, n=0; 10 μg/ml, 7.30±0.84 mm, n=5; 20 μg, 7.57±0.54 mm, n=5; 200 μg, 6.46±0.27 mm, n=5) produced a decrease in starting stretch length of the constructs, with no significant decrease at each concentration of aprotinin (FIG. 37). The construct starting stretch lengths were higher at all aprotinin concentrations for the pulsed as compared to the non-pulsed constructs. The altered pulsation group (1/12 pulsation) (2 wks, 6.20±0.20 mm, n=5), represented a value midway between the non-pulsed and pulsed constructs at 20 μg/ml aprotinin for starting stretch length (FIG. 38). Considering the starting stretch lengths of the constructs over time (1, 2, 3, 4, and 8 weeks), there was no significant difference. However, there was a significant difference between the non-pulsed and pulsed at all time points after 1 week at 20 μg/ml aprotinin,: (Non-pulsed: 1 wk, 5.95±0.05 mm, n=2; 2 wk, 4.94±0.38 mm, n=6; 3 wk, 5.70±0.50, n=2; 4 wk, 5.10±0.06 mm, n=3; 8 wk, 5.38±0.28 mm, n=4; Pulsed: 1 wk, 6.40±0.30 mm, n=2; 2 wk, 7.57±0.54 mm, n=5; 3 wk, 7.15±0.05, n=2; 4 wk, 7.50±0.5 mm, n=3; 8 wk, 7.43±0.15 mm, n=3) (FIG. 39).

### Example 28

### Stretch Length at Breaking Tension

Vessel constructs were step-wise stretched with known forces. Comparable lengths were then measured for each tension. When maximal breaking tension was applied to the constructs, the resulting stretch length was recorded. This represented a degree of elasticity at the constructs' maximal breaking tension. This elasticity was compared between various culture conditions and aprotinin concentrations.

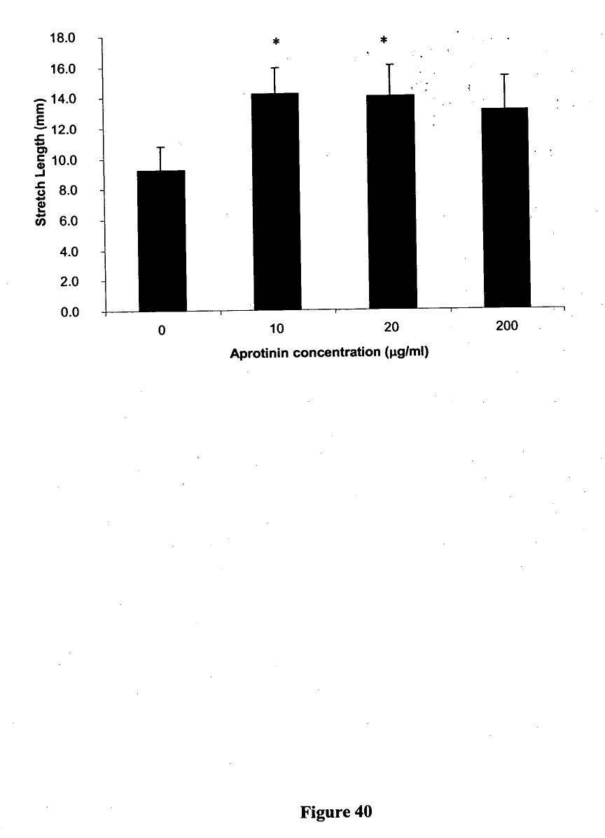

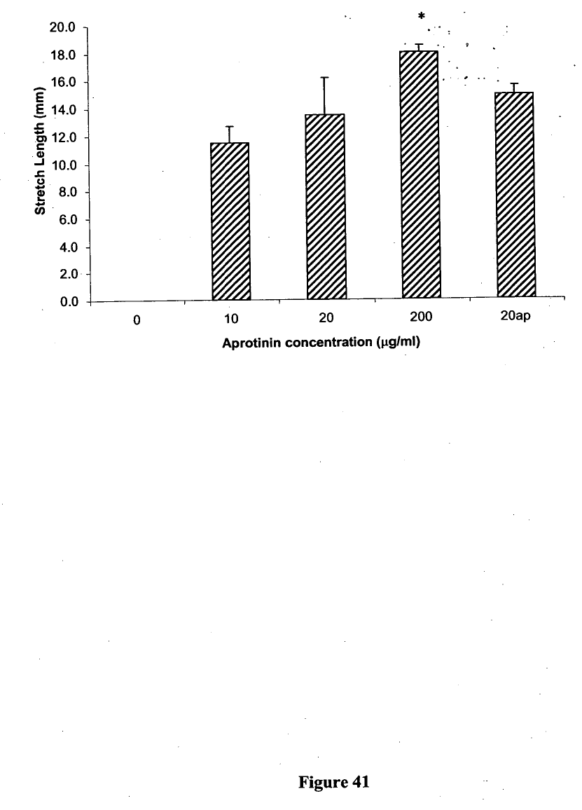

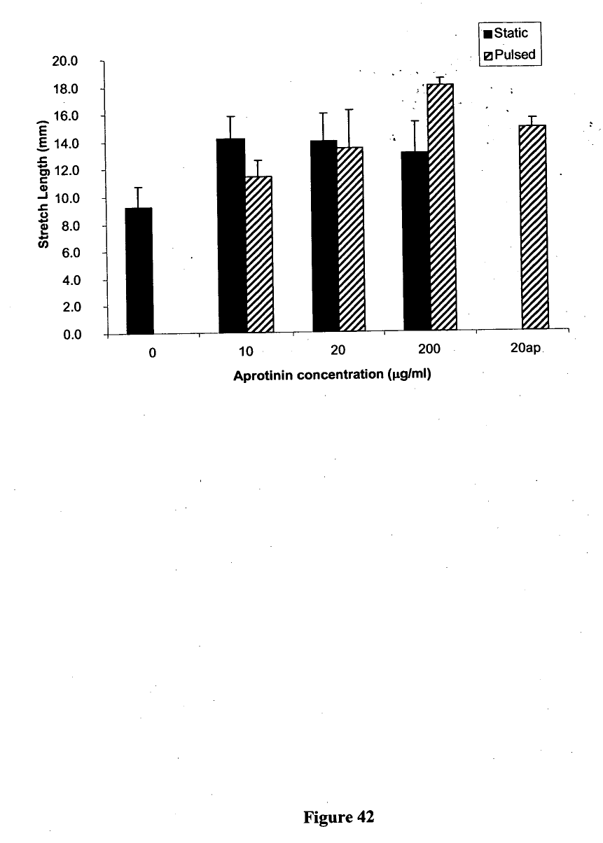

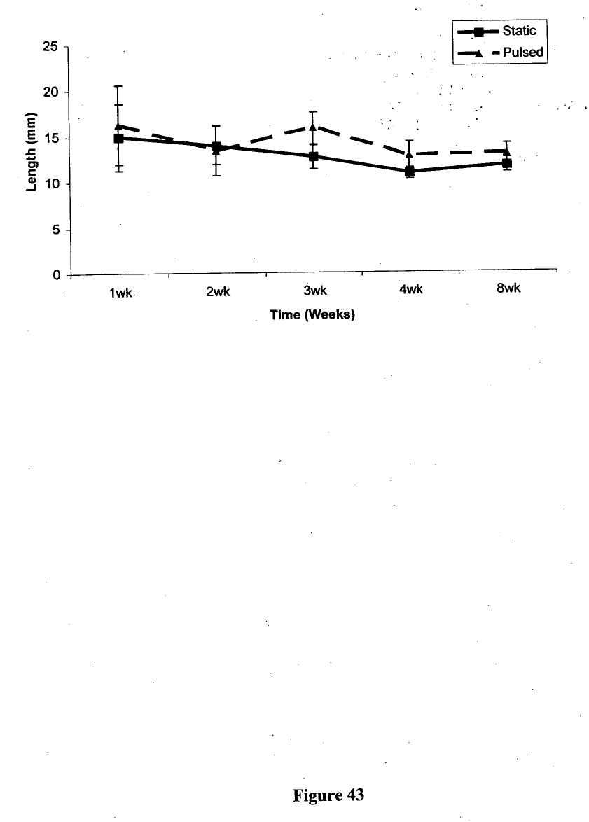

The non-pulsed fibrin gel constructs at 14 days demonstrated a significant increase in breaking length from 0 to 10 μg/ml aprotinin, and a steady breaking length after that (0 μg/ml, 9.23±1.53 mm, n=3; 10 μg/ml, 14.23±1.66 mm, n=4; 20 μg, 14.00±2.05 μg/mg, n=6; 200 μg, 13.05±2.26 mm, n=4) (FIG. 40). The pulsed fibrin gel constructs at 14 days also demonstrated that higher concentrations of aprotinin (0 μg/ml, 0.0±0.0 mg, n=0; 10 μg/ml, 11.44±1.19 mm, n=5; 20 μg, 13.45±2.76 mm, n=5; 200 μg, 17.98±0.51 mm, n=5) produced a steady increase in breaking length of the constructs, which was significant at 200 μg/ml aprotinin (FIG. 41). The construct breaking lengths were similar at all aprotinin concentrations for the pulsed as compared to the non-pulsed constructs at each concentration of aprotinin. The altered pulsation group (1/12 pulsation) (2 wks, 14.88±0.69 mm, n=5) represented a value similar to the non-pulsed and pulsed constructs at 20 μg/ml aprotinin for breaking length (FIG. 42). Considering the break lengths of the constructs over time (1, 2, 3, 4, and 8 weeks), there was no significant difference between the non-pulsed and pulsed at all time points (Non-pulsed: 1 wk, 14.95±3.65 mm, n=2; 2 wk, 14.00±2.05 mm, n=6; 3 wk, 12.70±1.30 mm, n=2; 4 wk, 10.87±0.96 mm, n=3; 8 wk, 11.70±0.81 mm, n=4; Pulsed: 1 wk, 16.25±4.35 mm, n=2; 2 wk, 13.45±2.76 mm, n=5; 3 wk, 15.85±1.75, n=2; 4 wk, 12.77±1.57 mm, n=3; 8 wk, 12.90±1.15 mm, n=3) (FIG. 43).

### Example 29

### Length Tension Curve

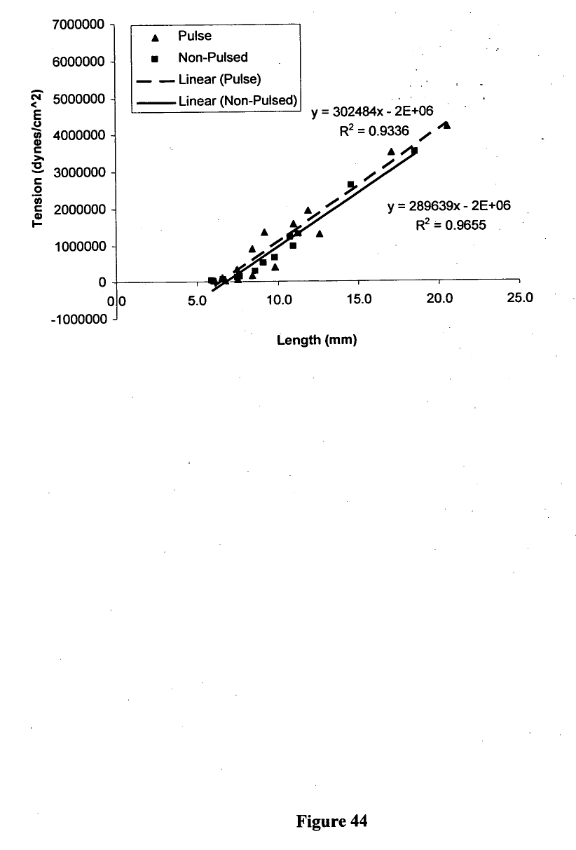

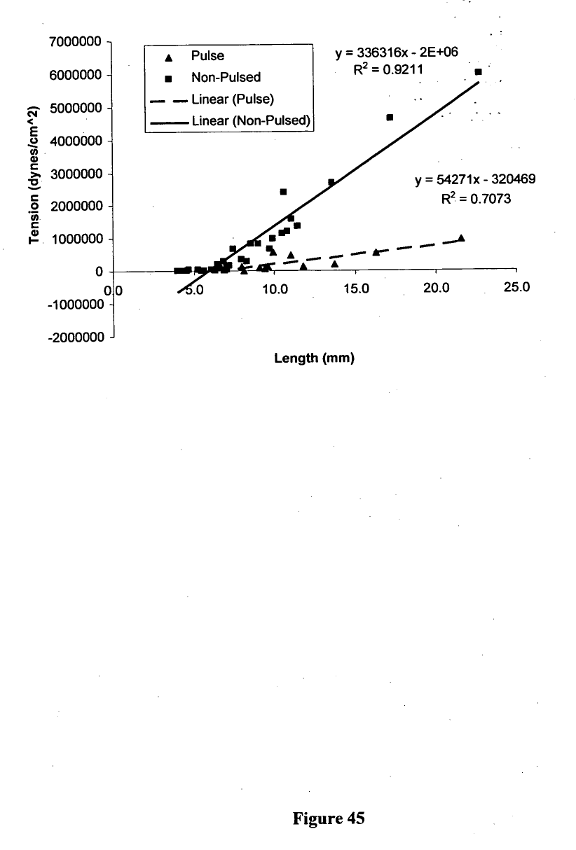

The tensile modulus of the fibrin gel constructs was measured by increasing the applied tension and recording the stretched length at each point. Length tension curve is a function of elasticity and strength. The tensile strength at 1 week culture time of the fibrin gel constructs comparing pulsed to non-pulsed showed a similar tensile strength (Pulsed: slope=3.0×10⁵, R=0.934; Non-pulsed: slope=2.9×10⁵, R=0.966) (FIG. 44). However, at 2 weeks of culture time, the length tension curve demonstrated that the non-pulsed constructs increased in tensile strength (slope=3.4×10⁵, R=0.921) and the pulsed constructs significantly decreased in tensile strength (slope=5.4×10⁴, R=0.707) (FIG. 45).

### Example 30

### Maximal Tensile Strength

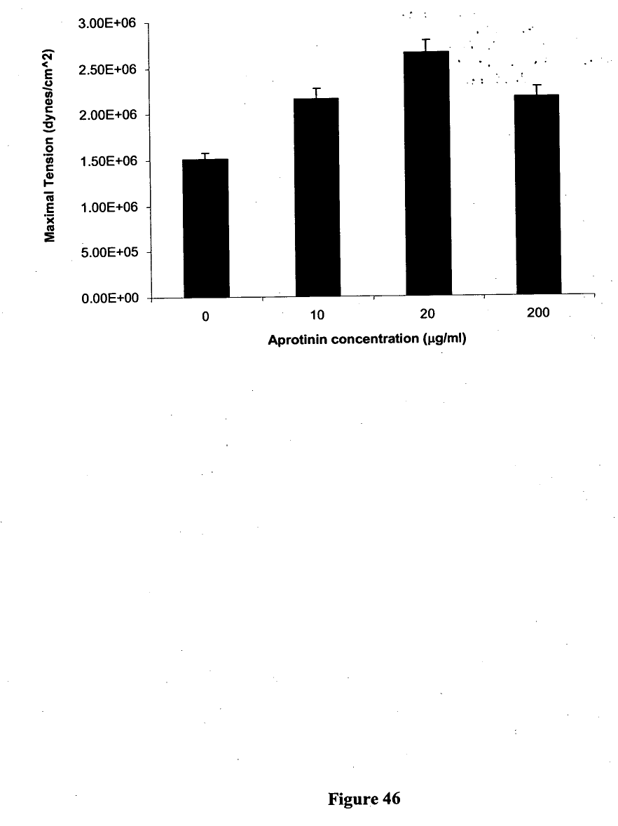

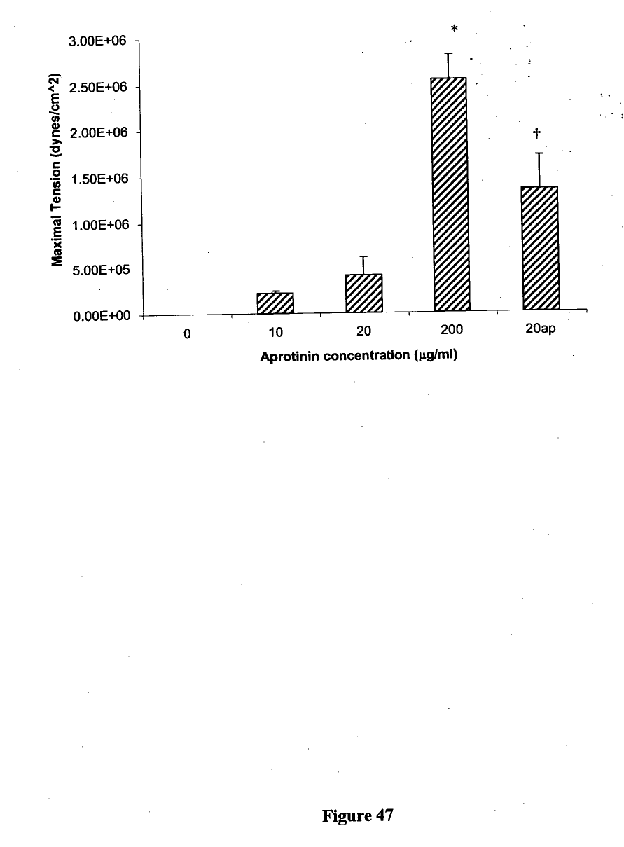

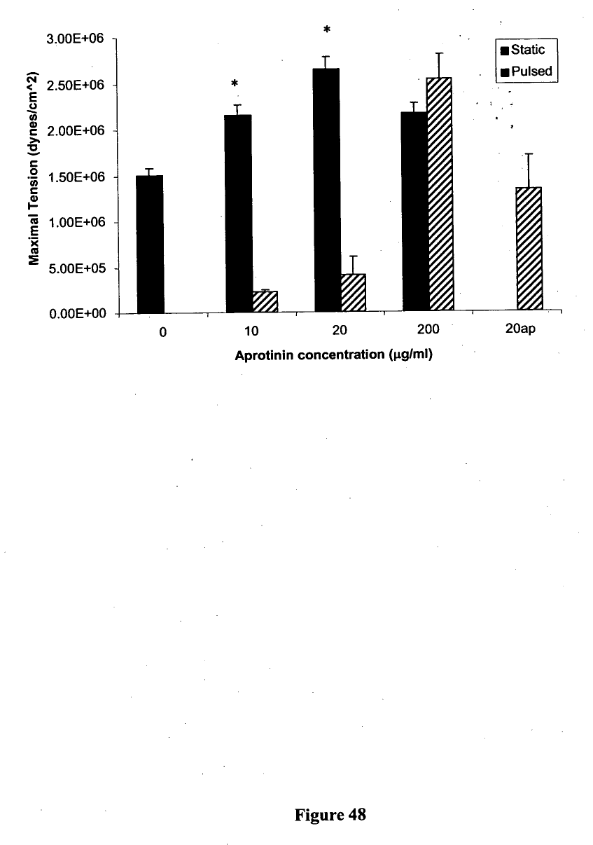

The test of maximal vessel strength was measured by applying a force to the inner lumen of the tissue ring while mounted in the isolated tissue bath. This force was applied until the tissue broke and the force was calculated as dynes/cm². As aprotinin concentrations increased from 0 to 200 μg/ml, the maximum tensile strength also increased in both the pulsed and the non-pulsed at 2 weeks, except at 200 μg/ml in the non-pulsed constructs, there was a small decrease in maximal tension (Non-pulsed: 0 μg/ml, 1.51×10⁶±1.10×10⁵ dynes/cm², n=3; 10 μg/ml, 2.16×10⁶±4.31×10⁵ dynes/cm², n=4; 20 μg/ml, 2.65×10⁶±8.60×10⁵ dynes/cm², n=6; 200 μg/ml, 2.17×10⁶±7.95×10⁵ dynes/cm², n=4; Pulsed: 10 μg/ml, 2.23×10⁵±2.41×10⁴ dynes/cm², n=5; 20 μg/ml, 4.09×10⁵±2.02×10⁵ dynes/cm², n=5; 200 μg/ml, 2.54×10⁶±2.70×10⁵ dynes/cm², n=5) (FIG. 46 and FIG. 47). At 0, 10, and 20 μg/ml aprotinin, the non-pulsed fibrin gel constructs demonstrated a much greater maximal tensile strength than the pulsed group. However, at 200 μg/ml aprotinin, the pulsed vessel was similar to that of the non-pulsed vessel (FIG. 48). The altered pulsation group (1/12 pulsation) (2 wks, 1.34×10⁶±3.74×10⁵ dynes/cm², n=5) represented a value midway between the non-pulsed and pulsed constructs at 20 μg/ml aprotinin.

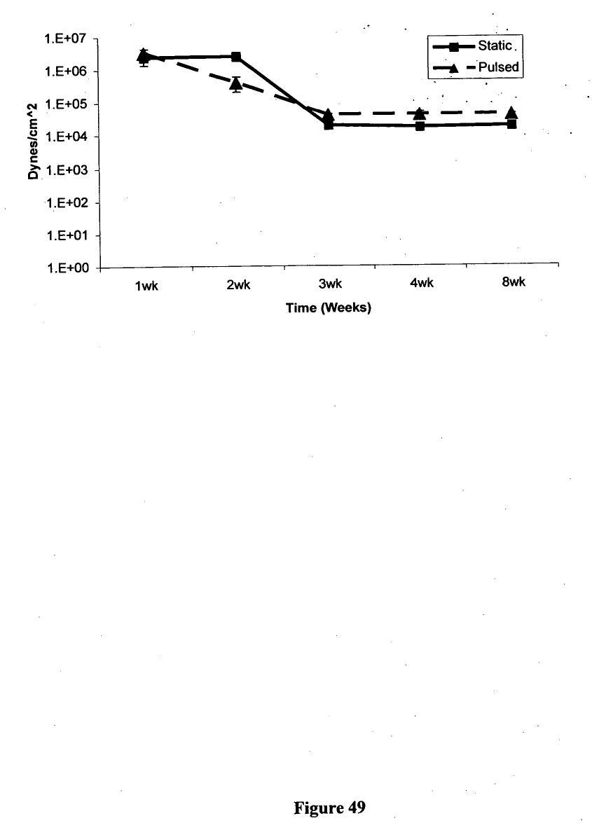

At 1 week, the break tensions of both the pulsed and non-pulsed tissues were similar (Non-pulsed: 20 μg/ml, 2.40×10⁶±1.10×10⁶ dynes/cm², n=2; Pulsed: 20 μg/ml, 3.07×10⁶±1.13×10⁶ dynes/cm², n=2). The breakpoints at 2 weeks were greatly different, but not significantly different (Non-pulsed: 20 μg/ml, 2.65×10⁶±8.60×10⁵ dynes/cm, n=6; Pulsed: 20 μμg/ml, 4.09×10⁵±2.02×10⁵ dynes/cm², n=5). At 3, 4, and 8 weeks, there was little difference between groups, and no change within groups overtime (FIG. 49).

### Example 31

### In-Vivo Vascular Grafting

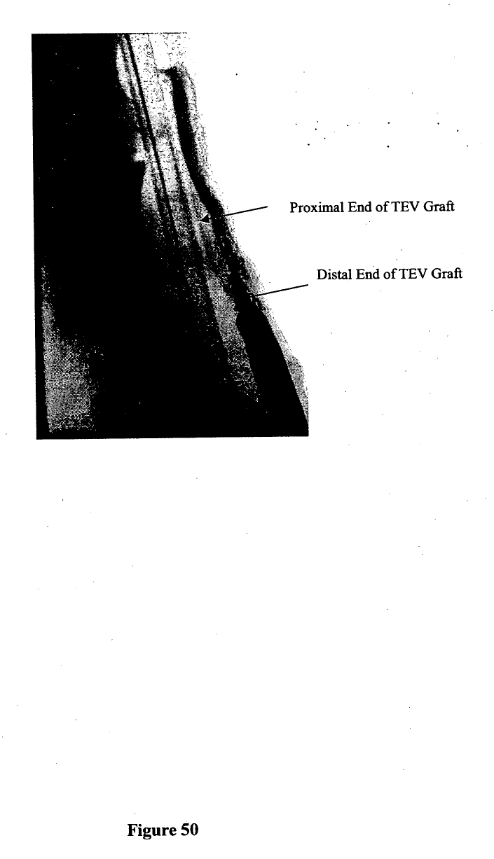

The optimal fibrin vessel construct parameters chosen to be used for an in-vivo vascular graft was non-pulsed and 20 μg/ml aprotinin. These constructs were implanted into the external jugular vein of a 12 week old lamb and left for 4 weeks to integrate. The first attempt was placed as a veinous patch. The construct covered approximately a half centimeter square area. The construct was doubled for added strength, and endothelial cells were seeded to the outer surface 3 days prior to grafting. An angiogram was done at 5 weeks to confirm patency and anatomical position. Also at 5 weeks, the vessel graft was removed and analyzed (FIG. 50). Histological sections were taken for hematoxylin and eosin staining as well as Mason's trichrome and Miller's elastin stain.

Following the successful grafting of the vein patch, additional animals were then grafted with similar constructs as interpositional vein grafts in the external jugular vein as well. These animals were followed at 4 weeks with an angiogram and subsequent ultrasound to confirm continued patency.

Although preferred embodiments have been depicted and described in detail herein, it will be apparent to those skilled in the relevant art that various modifications, additions, substitutions, and the like can be made without departing from the spirit of the invention and these therefore are considered within the scope of the invention as defined in the claims which follow.

What is claimed:

1. A method of producing a tissue-engineered vascular vessel comprising:
providing a vessel-forming fibrin mixture comprising fibrinogen, thrombin, and cells suitable for forming a vascular vessel;
molding the vessel-forming fibrin mixture into a fibrin gel having a tubular shape; and
incubating the fibrin gel having a tubular shape in a medium suitable for growth of the cells under conditions effective to produce a tissue-engineered vascular vessel.

2. The method according to claim 1, wherein the cells suitable for forming a vascular vessel are vascular smooth muscle cells.

3. The method according to claim 1, wherein the cells suitable for forming a vascular vessel are fibroblasts.

4. The method according to claim 1, wherein the cells suitable for forming a vascular vessel are in a concentration within the vessel-forming fibrin mixture of about 1 to 4×10⁶ cells/ml.

5. The method according to claim 1 further comprising:
controlling degradation rate of the vessel by addition of a protease inhibitor to the vessel-forming fibrin mixture.

6. The method according to claim 5, wherein the protease inhibitor is aprotinin.

7. The method according to claim 5, wherein the protease inhibitor is epsilonaminocaproic acid.

8. The method according to claim 1, wherein said molding is carried out in a tube with an inner mandrel.

9. The method according to claim 8, wherein the vessel has an interior surface, said method further comprising:
seeding endothelial cells on the interior surface of the vessel.

10. The method according to claim 1 further comprising:
subjecting the fibrin gel having a tubular shape to a pulse after said molding.

11. The method according to claim 1, wherein the medium suitable for growth comprises a growth additive.

12. The method according to claim 11, wherein the growth additive comprises a growth hormone selected from the group consisting of VEGF, b-FGF, PDGF, and KGF.

13. The method according to claim 1 further comprising:
changing the medium suitable for growth.

14. The method according to claim 1, wherein the vessel has an outer surface to which cells are added during said molding.

15. The method according to claim 14, wherein the cells to be added to the outer surface of the vessel are fibroblasts.

16. The method according to claim 14, wherein the cells to be added to the outer surface of the vessel are specific organ cells.

17. The method according to claim 1, wherein the fibrin gel is combined with a porous scaffold to enhance vascular grafting.

18. The method according to claim 17, wherein the porous scaffold is decellularized elastin.

19. The method according to claim 17, wherein the porous scaffold is poly lactic-glycolic acid.

20. A tissue-engineered vascular vessel produced by the method of claim 1.

21. A tissue-engineered vascular vessel comprising:
a gelled fibrin mixture comprising fibrinogen, thrombin, and cells, wherein the gelled fibrin mixture has a tubular shape.

22. The tissue-engineered vascular vessel according to claim 21, wherein the cells are vascular smooth muscle cells.

23. The tissue-engineered vascular vessel according to claim 21, wherein the cells are fibroblasts.

24. The tissue-engineered vascular vessel according to claim 21, wherein the cells are in a concentration in the gelled fibrin mixture of about 1 to 4×10⁶ cells/ml.

25. The tissue-engineered vascular vessel according to claim 21, wherein the gelled fibrin mixture further comprises a protease inhibitor.

26. The tissue-engineered vascular vessel according to claim 25, wherein the protease inhibitor is aprotinin.

27. The tissue-engineered vascular vessel according to claim 25, wherein the protease inhibitor is epsilonaminocaproic acid.

28. The tissue-engineered vascular vessel according to claim 21, wherein the vessel has an interior surface on which endothelial cells are present.

29. The tissue-engineered vascular vessel according to claim 21, wherein the vessel has an outer surface on which cells are present.

30. The tissue-engineered vascular vessel according to claim 29, wherein the cells present on the outer surface of the vessel are fibroblasts.

31. The tissue-engineered vascular vessel according to claim 29, wherein the cells present on the outer surface of the vessel are specific organ cells.

32. The tissue-engineered vascular vessel according to claim 21, wherein the gelled fibrin mixture contains a porous scaffold.

33. The tissue-engineered vascular vessel according to claim 32, wherein the porous scaffold is decellularized elastin.

34. The tissue-engineered vascular vessel according to claim 32, wherein the porous scaffold is poly lactic-glycolic acid.

35. A method of producing a tissue-engineered vascular vessel for a particular patient comprising:
providing a vessel-forming fibrin mixture comprising fibrinogen, thrombin, and cells suitable for forming a vascular vessel, at least one of which is autologous to the patient;
molding the vessel-forming fibrin mixture into a fibrin gel having a tubular shape;
incubating the fibrin gel having a tubular shape in a medium suitable for growth of the cells under conditions effective to produce a tissue-engineered vascular vessel for a particular patient; and
implanting the tissue-engineered vascular vessel into the particular patient.

36. The method according to claim 35, wherein the fibrinogen is autologous.

37. The method according to claim 35, wherein the cells suitable for forming a vascular vessel are vascular smooth muscle cells.

38. The method according to claim 35, wherein the cells suitable for forming a vascular vessel are fibroblasts.

39. The method according to claim 35, wherein the cells suitable for forming a vascular vessel are present in the vessel-forming fibrin mixture in a concentration of about 1 to 4×10⁶ cells/ml.

40. The method according to claim 35, wherein the cells suitable for forming a vascular vessel are autologous.

41. The method according to claim 35 further comprising:
controlling degradation rate of the vessel by addition of a protease inhibitor to the vessel-forming fibrin mixture.

42. The method according to claim 41, wherein the protease inhibitor is aprotinin.

43. The method according to claim 41, wherein the protease inhibitor is epsilonaminocaproic acid.

44. The method according to claim 35, wherein said molding is carried out in a tube with an inner mandrel.

45. The method according to claim 44, wherein the vessel has an interior surface, said method further comprising:
seeding endothelial cells on the interior surface of the vessel.

46. The method according to claim 35 further comprising:
subjecting the fibrin gel having a tubular shape to a pulse after said molding.

47. The method according to claim 35, wherein the medium suitable for growth comprises a growth additive.

48. The method according to claim 47, wherein the growth additive comprises a growth hormone selected from the group consisting of VEGF, b-FGF, PDGF, and KGF.

49. The method according to claim 35 further comprising:
changing the medium suitable for growth.

50. The method according to claim 35, wherein the vessel has an outer surface to which cells are added during said molding.

51. The method according to claim 50, wherein the cells to be added to the outer surface of the vessel are fibroblasts.

52. The method according to claim 50, wherein the cells to be added to the outer surface of the vessel are specific organ cells.

53. The method according to claim 35, wherein the fibrin gel is combined with a porous scaffold to enhance said implanting.

54. The method according to claim 53, wherein the porous scaffold is decellularized elastin.

55. The method according to claim 53, wherein the porous scaffold is poly lactic-glycolic acid.

56. A tissue-engineered vascular vessel produced by the method of claim 35.
